SEQUENCE LISTING (5 sequences)

SEQ ID NO: 1 - DNA, 1687 bp, Artificial Sequence
  Description of Artificial Sequence Mouse-Human Hybrid Gene
aagcttatggattttcaagtgcagattttcagcttcctgctaatcagtgcttcagtcataatgtccagaggagtcgacatccagatgacacagactacatcctccctgtctgcctctctgggagacagagtcaccatcagttgcagggcaagtcaggacattcgcaattatttaaactggtatcagcagaaaccagatggaactgttaaactcctgatctactacacatcaagattacactcaggagtcccatcaaggttcagtggcagtgggtctggaacagattattctctcaccattgccaacctgcaaccagaagatattgccacttacttttgccaacagggtaatacgcttccgtggacgttcggtggaggcaccaaactggtaaccaaacgggagctcggtggcggtggctcgggcggtggtgggtcgggtggcggcggatctatcgatgaggtccagctgcaacagtctggacctgaactggtgaagcctggagcttcaatgtcctgcaaggcctctggttactcattcactggctacatcgtgaactggctgaagcagagccatggaaagaaccttgagtggattggacttattaatccatacaaaggtcttactacctacaaccagaaattcaagggcaaggccacattaactgtagacaagtcatccagcacagcctacatggagctcctcagtctgacatctgaagactctgcagtctattactgtgcaagatctgggtactatggtgactcggactggtacttcgatgtctggggcgcagggaccacggtcaccgtctcctctgatctggagcccaaatcttctgacaaaactcacacaagcccaccgagcccagcacctgaactcctggggggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagcggatccttcgaacctgctcccatcctgggccattaccttaatctcagtaaatggaatttttgtgatatgctgcctgacctactgctttgccccaagatgcagagagagaaggaggaatgagagattgagaagggaaagtgtacgccctgtataaatcgatactcgag

SEQ ID NO: 2 - DNA, 1696 bp, Artificial Sequence
  Description of Artificial Sequence Mouse-Human Hybrid Gene
aagcttgccgccatgaggttctctgctcagcttctggggctgcttgtgctctggatccctggatccactgcagatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttccgctcacgttcggtgctgggaccaagctggagctgaaacggggtggcggtggctcgggcggtggtgggtcgggtggcggcggatcgtcacaggtgcagctgaagcagtcaggacctggcctagtgcagtcctcacagagcctgtccatcacctgcacagtctctggtttctcattaactacctatgctgtacactgggttcgccagtctccaggaaagggtctggagtggctgggagtgatatggagtggtggaatcacagactataatgcagctttcatatccagactgagcatcaccaaggacgattccaagagccaagttttctttaaaatgaacagtctgcaacctaatgacacagccatttattactgtgccagaaatgggggtgataactacccttattactatgctatggactactggggtcaaggaacctcagtcaccgtctcctctgatctggagcccaaatcttctgacaaaactcacacaagcccaccgagcccagcacctgaactcctggggggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagcggatccttcgaacctgctcccatcctgggccattaccttaatctcagtaaatggaatttttgtgatatgctgcctgacctactgctttgccccaagatgcagagagagaaggaggaatgagagattgagaagggaaagtgtacgccctgtataaatcgatactcgag

SEQ ID NO: 3 - protein, 555 aa, Artificial Sequence
  Description of Artificial Sequence Mouse-Human Fusion Protein
MDFQVQIFSFLLISASVIMSRGVDIQMTQTTSSLSASLGDRVTISCRASQDIRNYLNWYQQKPDGTVKLLIYYTSRLHSGVPSRFSGSGSGTDYSLTIANLQPEDIATYFCQQGNTLPWTFGGGTKLVTKRELGGGGSGGGGSGGGGSIDEVQLQQSGPELVKPGASMSCKASGYSFTGYIVNWLKQSHGKNLEWIGLINPYKGLTTYNQKFKGKATLTVDKSSSTAYMELLSLTSEDSAVYYCARSGYYGDSDWYFDVWGAGTTVTVSSDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKADPSNLLPSWAITLISVNGIFVICCLTYCFAPRCRERRRNERLRRESVRPV

SEQ ID NO: 4 - protein, 556 aa, Artificial Sequence
  Description of Artificial Sequence Mouse-Human Fusion Protein
MRFSAQLLGLLVLWIPGSTADIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPLTFGAGTKLELKRGGGGSGGGGSGGGGSSQVQLKQSGPGLVQSSQSLSITCTVSGFSLTTYAVHWVRQSPGKGLEWLGVIWSGGITDYNAAFISRLSITKDDSKSQVFFKMNSLQPNDTAIYYCARNGGDNYPYYYAMDYWGQGTSVTVSSDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKADPSNLLPSWAITLISVNGIFVICCLTYCFAPRCRERRRNERLRRESVRPV

SEQ ID NO: 5 - DNA, 55 bp, Artificial Sequence
  Description of Artificial Sequence Primer
cgtcgatgagctctagaattcgcatgtgcaagtccgatgagtccccccccccccc

(19) United States
(12) Patent Application Publication
(10) Pub. No.: US 2004/0058445 A1
(43) Pub. Date: Mar. 25, 2004

(54) Activation of tumor-reactive lymphocytes via antibodies or genes recognizing CD3 or 4-1BB

(76) Inventors: Jeffrey Alan Ledbetter, Shoreline, WA (US); Ingegerd Hellstrom, Seattle, WA (US); Martha Hayden-Ledbetter, Shoreline, WA (US); Karl Erik Hellstrom, Seattle, WA (US)

Correspondence Address: Bradford J. Duft, Esq., Buchanan Ingersoll Professional Corporation, One Oxford Centre, 301 Grant Street, Pittsburgh, PA, 15219-1410, US

(21) Appl. No.: 10/107,991

(22) Filed: Mar. 26, 2002

Related U.S. Application Data
(60) Provisional application No. 60/286,585, filed on Apr. 26, 2001.

(51) Int. Cl. C07H 21/04; C12N 5/08; C12P 21/02; C07K 16/30

(52) U.S. Cl. 435/372; 435/69.1; 435/320.1; 530/388.8; 536/23.53

(57) ABSTRACT

An improved method for ex-vivo generation of tumor-reactive T cells is provided, comprising culturing PBMC from a patient with cancer with autologous tumor cells and magnetic beads which activate T lymphocytes via CD3 in combination with CD28, CD40, or CD28 plus CD40. The invention also provides genes encoding anti-CD3 or anti-4-1BB scFv and methods to transfect cells of neoplastic or normal origin for expression of those genes at their surface. The genes and transfected tumor cells are useful compositions for induction of a tumor-destructive immune response.

### CROSS-REFERENCE TO RELATED APPLICATIONS

This patent application claims the benefit of provisional patent application No. 60/286,585, filed on Apr. 26, 2001.

### STATEMENT REGARDING FEDERALLY SPONSORED RESEARCH

Portions of this work were funded by grants from the United States National Institutes of Health, and the U.S. government has rights in the invention.

### REFERENCE TO MICROFICHE APPENDIX

This application includes a sequence listing composed of four sequences for DNA and proteins claimed herein. The identity of the paper copy and the computer copies are identical.

### BACKGROUND OF THE INVENTION

Tumors have potential targets for immunotherapy. Human tumors express a variety of tumor antigens, most of which are present in some normal tissues, albeit at lower levels (Hellstrom and Hellstrom, Adv Cancer Res, 12, 167-223, 1969; Cheever et al., Immunol Rev, 145, 33-59, 1995; Finn et al., Immunol Rev, 145, 61-89, 1995; Boon et al., Immunol Today, 18, 267-8, 1997; Hellstrom and Hellstrom, Handbook of Experimental Pharmacology, Vaccines (Chapter 17), 463-478, 1999). Many of these antigens are immunogenic in the tumor-bearing host. For example, IgG antibodies to a variety of tumor-associated antigens can be detected by the SEREX technique (Old and Chen, J. Exp. Med., 187, 1163-1167, 1998), and T cells recognizing tumor antigens can be demonstrated by using tetramers (Cassian et al., J.Immunol., 162, 1999), as well as by the ability to generate tumor-selective T cell clones in vitro (Boon, Coulie et al., Immunol Today, 18, 267-8, 1997). This provides an impetus for various forms of immunotherapy, including the administration of in vitro expanded immune T lymphocytes (Rosenberg, Biologic Therapy of Cancer (Chapter 19), 487, 1995) and therapeutic tumor vaccination (Nestle et al., Nat Med, 4, 328-32, 1998; Rosenberg et al., Nat Med, 4, 321-7, 1998) (Greenberg, Adv Immunol, 49, 281-355, 1991; Melief and Kast, Immunol Rev, 145, 167-77, 1995),(Pardoll, Curr Opin Immunol, 8, 619-21, 1996),(Hellstrom and Hellstrom, Handbook of Experimental Pharmacology, Vaccines (Chapter 17), 463-478, 1999).

Mouse tumors provide useful models towards developing more effective immunotherapy, since they express targets for a tumor destructive immune response, although certain experimental manipulations are needed to obtain an effective immune response against most tumors of spontaneous origin (Greenberg, Adv Immunol, 49, 281-355, 1991; Kerr and Mule', J. Leuko. Biol., 56, 210-214, 1994; Cheever, Disis et al., Immunol Rev, 145, 33-59, 1995; Finn, Jerome et al., Immunol Rev, 145, 61-89, 1995; Melief and Kast, Immunol Rev, 145, 167-77, 1995; Pardoll, Curr Opin Immunol, 8, 619-21, 1996; Boon, Coulie et al., Immunol Today, 18, 267-8, 1997; Hellstrom and Hellstrom, Handbook of Experimental Pharmacology, Vaccines (Chapter 17), 463-478, 1999).

Induction of anti-tumor immunity. T lymphocytes (CD8+ and CD4+) play a key role in the generation (and commonly also the execution) of a tumor-destructive response, but NK cells and antibodies also contribute, as do macrophages (Hellstrom and Hellstrom, Adv Cancer Res, 12, 167-223, 1969; Greenberg, Adv Immunol, 49, 281-355, 1991; Melief and Kast, Immunol Rev, 145, 167-77, 1995; Hellstrom and Hellstrom, Handbook of Experimental Pharmacology, Vaccines (Chapter 17), 463-478, 1999). Antigen presentation is normally by dendritic cells (DC) (Huang et al., Science, 264, 961-5, 1994) which are differentiated from stem cells in the bone marrow and monocytes in the blood, but can, under certain circumstances also be accomplished by the tumor cells themselves (Chen et al., Cell, 71, 1093-102, 1992; Schoenberger et al., Cancer Res, 58, 3094-100, 1998). Procedures facilitating the presentation of tumor antigens by DC are crucial to obtain effective tumor immunity, and there are recent data indicating that they can make possible a more effective therapy of certain human cancers (see, e.g., (Kugler et al., Nature Medicine, 6, 332-, 2000)). A combination of CD8+ T lymphocytes with in vitro CTL activity and lymphokine-producing T helper cells are needed in the rejection of most tumors (Hellstrom and Hellstrom, Handbook of Experimental Pharmacology, Vaccines (Chapter 17), 463-478, 1999). Although both Th1 and Th2 responses can be favorable (Rodolfo et al., J Immunol, 163, 1923-8, 1999), the Th1 responses play the dominant role in the immune destruction of tumors (Hu et al., J Immunol, 161, 3033-41, 1998).

To induce an immune response, costimulation, particularly by interaction between CD80 and/or CD86 on the APC and CD28 on the T lymphocytes, is necessary (Schwartz, Cell, 57, 1073-81, 1989; June et al., Immunol Today, 11, 211-6, 1990; Linsley and Ledbetter, Annu Rev Immunol, 11, 191-212, 1993). It leads to the sustained production of IL2, IFN-γ, and other lymphokines needed to expand an immune response (Thompson et al., Proc Natl Acad Sci U S A, 86, 1333-7, 1989) and serves a similar purpose as using tumor cells transfected with genes encoding lymphokines (Pardoll, Curr Opin Immunol, 8, 619-21, 1996). Without a second signal via CD28, exposure of the TCR to antigen does not induce an immune response, and it can even induce anergy. Since most tumors do not express CD80 or CD86 (Chen, Ashe et al., Cell, 71, 1093-102, 1992; Yang et al., J Immunol, 154, 2794-800, 1995), no effective immunity is induced until antigen has reached the tumor-draining lymph nodes and been taken up, processed and presented by DC, which express CD80 and CD86 (Huang, Golumbek et al., Science, 264, 961-5, 1994; Yang et al., J Immunol, 158, 851-8, 1997). This may explain why tumors often “sneak through” the immune system until there is an established tumor mass. CD80 and CD86 bind not only to CD28 but with even higher avidity to CTLA4 on activated T cells. The latter binding induces a negative signal which can terminate the immune response (Thompson, Lindsten et al., Proc Natl Acad Sci U S A, 86, 1333-7, 1989; Walunas et al., Immunity, 1, 405-13, 1994; Krummel and Allison, J Exp Med, 183, 2533-40, 1996; Leach et al., Science, 271, 1734-6, 1996; Walunas et al., J Exp Med, 183, 2541-50, 1996; Allison et al., Novartis Found Symp, 215, 92-8, 1998) and indicates that procedures engaging CD28 but not CTLA4 have therapeutic advantage.

The immune system is relatively ineffective in destroying established tumors. Immune responses, as induced by conventional tumor vaccines or following the transfer of immune T cells with in vitro anti-tumor activity, are rarely capable of destroying more than a few million tumor cells. Several escape mechanisms have been identified which may be responsible for this since they can protect tumors from an immunological attack (Hellstrom and Hellstrom, Handbook of Experimental Pharmacology, Vaccines (Chapter 17), 463-478, 1999; Kiessling et al., Cancer Immunol. Immunother., 48, 353-362, 1999). They include loss of tumor epitopes (Maeurer et al., J Clin Invest, 98, 163-341, 1996), and/or of MHC class I molecules which can present such epitopes to CTL [Restifo, 1993 #197; Maeurer, 1996 #403; Hellstrom, 1997 #58; Garrido, 1997 #220; Johnsen, 1998 #121, and elimination of tumor-reactive lymphocytes apoptosis (Hahne et al., Science, 274, 1363-1366, 1996; Shiraki et al., Proc Natl Acad Sci U S A, 94, 6420-5, 1997; Bennett et al., J Immunol, 160, 5669-75, 1998; Kume et al., Int J Cancer, 84, 339-43, 1999) (Chappell and Restifo, Cancer Immunol Immunother, 47, 65-71, 1998). However, tumors which can present immunogenic tumor antigens and do not induce apoptosis of reactive lymphocytes commonly escape from immune control. This has been attributed to various “blocking factors” produced by either the tumor, the host or both and acting directly on the T cells or indirectly via APC with or without epitope selectivity. They include, soluble tumor antigen and immune complexes, as well as TGF-beta, prostaglandins, NO etc (Hellstrom and Hellstrom, Adv Immunol, 18, 209-77, 1974; Kehrl et al., J Exp Med, 163, 1037-50, 1986; Sulitzeanu, Adv Cancer Res, 60, 247-267, 1993; Kiessling et al., Springer Semin. Immunopathol., 18, 227-242, 1996; Kiessling, Wasserman et al., Cancer Immunol. Immunother., 48, 353-362, 1999). Downregulation by antigen released from tumor cells, alone or in combination with antibodies as an immune complex may occur by deviating a tumor-destructive Th1 response (Hu, Urba et al., J Immunol, 161, 3033-41, 1998) to a Th2 response (Fiorentino et al., J Exp Med, 170, 2081-95, 1989) and may be accompanied by the production of TGFβ, which can be secondarily to the production of Th2 lymphokines such as IL-10 (Wilbanks et al., Eur J Immunol, 22, 165-73, 1992; D'Orazio and Niederkorn, J Immunol, 160, 2089-98, 1998). Downregulation may also be due to interaction between CTLA4 on activated T lymphocytes and CD80/CD86 on APC or activated T cells (Leach, Krummel et al., Science, 271, 1734-6, 1996; Allison, Chambers et al., Novartis Found Symp, 215, 92-8, 1998), and this may also be mediated by TGF-β (Chen et al., J Exp Med, 188, 1849-57, 1998). A downregulatory role of macrophages, producing NO and Prostaglandins has been identified as well (Kiessling, Wasserman et al., Cancer Immunol. Immunother., 48, 353-362, 1999).

Inhibition of T cell reactivity is reflected by the finding that T cell signaling mechanisms are often defective among tumor-infiltrating T lymphocytes (Mizoguchi et al., Science, 258, 1795-8, 1992; Nakagomi et al., Cancer Res, 53, 5610-5612, 1993; Kiessling, Wasserman et al., Cancer Immunol. Immunother., 48, 353-362, 1999), and they can recover when the lymphocytes are removed from the body. Data that are summarized in Section C indicate that human patients with advanced cancer have low expression of CD3 (reflecting a low TCR expression). They also show that polyclonal T cell activation via CD3 can quickly restore the downregulated CD3 expression, expand already existing, tumor-reactive but “dormant” T cell populations and facilitate the generation of tumor-selective CTL.

It is noteworthy that there are a few situations when even large tumors have been rejected by the immune system. This is illustrated by findings when engaging the T cell activation molecule 4-1BB to treat mouse tumors (Melero et al., Nat Med, 3, 682-5, 1997; Melero et al., Eur J Immunol, 28, 1116-21, 1998).

However, the most striking example is probably the observation, during the early days of kidney transplantation, that some patients who had large metastases arising from cancer cells that had contaminated a cadaver transplant completely recovered following removal of the immunosuppression (Wilson et al., N Engl J Med, 278, 479-83, 1968; Matter et al., Transplantation, 9, 71-4, 1970).

Engagement of 4-1BB can induce tumor regression. The cell surface molecule 4-1BB is expressed on activated but not on naive T cells (DeBenedette et al., J Exp Med, 181, 985-92, 1995; Shuford et al., J Exp Med, 186, 47-55, 1997) and engaging 4-1BB is thus likely to amplify an immune response that has been already induced. Exposure to anti4-1BB MAbs can stimulate the proliferation of antigen-activated CD8+ T lymphocytes with CTL activity, as well as the production/release of IFN-γ and other cytokines of the Th1 type (IL-2, TNF-α), and it can protect T cells against apoptosis (Hurtado et al., J Immunol, 158, 2600-9, 1997; Kim et al., Eur J Immunol, 28, 881-90, 1998; Natoli et al., Biochem Pharmacol, 56, 915-20, 1998; Takahashi et al., J Immunol, 162, 5037-40, 1999; Tsushima et al., Exp Hematol, 27, 433-40, 1999). In addition, 4-1BB has an immunoregulatory effect that involves NK1.1 cells (Melero et al., Cell Immunol, 190, 167-72, 1998).

MAbs to 4-1BB can have dramatic activity against well-established (approximately 10 mm diameter) tumors in mice, including tumors of low immunogenicity and CD8+ CTL with increased cytolytic activity have been generated from lymphocytes of mice treated with anti-4-1BB MAb (Melero, Shuford et al., Nat Med, 3, 682-5, 1997). Exposure of lymphocytes to tumor cells transfected to incorporate the 4-1BB ligand (4-1BBL), which binds to 4-1BB, can also significantly expand CD8+ T cell responses, and 4-1BBL-transfected tumor cells have therapeutic activity when used as vaccines in mouse models. However, there is evidence that administration of anti4-1BB MAb is more effective than vaccination with tumor cells expressing the 4-1BBL, since antibody treatment is efficacious against the non-immunogenic sarcoma Ag104 (Melero, Shuford et al., Nat Med, 3, 682-5, 1997), while Ag104 cells transfected to express 4-1BBL need to be combined with such cells expressing CD80 in order to obtain a therapeutic effect against this tumor [Melero, 1998 #19

An alternative approach to increase tumor immunity may be to administer a dose of anti-CD3 Mab that will provide polyclonal T cell activation, including activation of the clones of any tumor-reactive lymphocytes, and some therapeutic success with this approach has been described in studies using a mouse model (Ellenhorn et al., Science, 242, 569-71, 1988).

Tumor-reactive T cells can be generated in vitro for in vivo use. Tumor immunity can be transferred with lymphocytes to prevent the outgrowth of transplanted cells from the respective neoplasm (Klein et al., Cancer Res, 20, 1561-1572, 1960), and rejection of small, established tumors following adoptive transfer of immune lymphocytes was demonstrated many decades ago (Hellstrom et al., Transplant Proc, 1, 90-4, 1969). Adoptively transferred lymphocytes localize preferentially to the tumors to which they have been immunized (Mule' et al., J. Immunol., 123, 600-606, 1979), a finding that has stimulated the use of in vitro expanded, tumor-infiltrating lymphocytes (TIL) for therapy (Rosenberg, Biologic Therapy of Cancer (Chapter 19), 487, 1995). Although dramatic clinical responses have been seen in a small fraction of patients, the degree of therapeutic success is often modest, both in mice carrying tumors larger than a few mm in diameter (Greenberg, Adv Immunol, 49, 281-355, 1991; Chen, Ashe et al., Cell, 71, 1093-102, 1992; Melief and Kast, Immunol Rev, 145, 167-77, 1995; Hellstrom and Hellstrom, Handbook of Experimental Pharmacology, Vaccines (Chapter 17), 463-478, 1999) and in man. The failures may have resulted from some of the previously discussed “escape” mechanisms and/or from faulty localization of the infused lymphocytes within the tumor mass.

Improved methods are therefore needed both to construct tumor vaccines that induce more robust immune responses and to generate T lymphocytes for therapy of cancer patients.

### SUMMARY

This invention relates to improved methods for the generation of tumor reactive T cells in vitro and to the composition of matter for tumor vaccines to be therapeutically used in vivo. A method is first described in which mononuclear lymphoid cells from peripheral blood or tumors are harvested from cancer patients and cultured with autologous tumor cells in the presence of immobilized antibodies specific for CD3 and CD28 over a 4-5 day period. Cells can be expanded to therapeutic useful levels in 10 ul/ml IL-2 after the beads with immobilized antibodies are removed. This method is useful for improved generation of tumor-reactive lymphocytes for therapy of cancer. While not being bound by theory, we believe that the invention operates through the following components: T lymphocytes, whose expression of CD3 is originally low, are polyclonally activated, proliferate vigorously, form Th1 type lymphokines and rapidly destroy the tumor cells, releasing tumor antigens. The polyclonal T cell activation also causes the maturation of monocytes in the cultures to dendritic cells, which take up dead tumor cells, process and present tumor antigens to induce the continued expansion of tumor-specific T cells, including CTL. The invention also provides genes encoding anti-CD3 or anti4-1BB single chain Fv (scFv) molecules expressed on the tumor cell surface and cells transfected with these genes for in vivo cancer therapy. The anti-CD3 scFv expression on the surface of tumor cells induces polyclonal T cell activation and tumor cell destruction, releasing tumor antigens and promotes a transition to antigen-specific tumor immunity, detected as rejection of “wild type” (not transfected) cells from the same tumor. Expression on the surface of tumor cells of the anti-4-1BB scFv induces activation/expansion of tumor-reactive T cells by increasing their proliferation and/or by protecting them from apoptosis, to cause the production of tumor-reactive lymphokines, such as IFN-gamma. After immunization with tumor cells transfected to express anti-4-1BB scFv on the cell surface, wild type cells from the same tumor are rejected by a mechanism involving activation of NK cells and CD4+ T cells. Tumor cells expressing anti-4-1BB scFv on the cell surface are active in therapy of established wild-type tumors.

The invention makes possible two novel methods of cancer therapy: First, it shows how to activate “suppressed” lymphocytes by immobilized anti-CD3/anti-CD28/anti-CD40 (or anti-CD3/CD28) beads so they proliferate, make Th1 lymphokines, become less sensitive to inhibition by TGF-beta. The activated lymphocytes destroy tumor cells thus providing tumor antigen while also inducing maturation of APC. This method leads over time to an expansion of tumor-reactive CD8+ and CD4+ T cells and NK cells that are better suited for adoptive transfer to cancer patients. Second, it shows that genes of the invention encoding anti-CD3 or anti4-1BB scFv at the tumor cell surface can effectively induce a tumor-destructive immune response against wild type cells from the same tumor.

### DRAWING FIGURES

1. Peripheral blood mononuclear cells (PBMC) and tumor-infiltrating lymphocytes (TIL) from a patient with advanced ovarian carcinoma proliferate in the presence of autologous tumor cells and beads stimulating CD3 in combination with CD28 and CD40.

2. Combination of autologous, but not allogeneic, tumor cells and beads that stimulate via CD3 in combination with CD28 induce proliferation of PBMC from a patient with colon carcinoma.

3. PBMC from a patient with colon carcinoma in the presence of beads that stimulate CD3 in combination with CD28 or both CD28 and CD40 lyse autologous tumor cells in a 4 hr Cr⁵¹ release assay.

4. PBMC from a patient with head and neck carcinoma produce IFN gamma following cultivation with autologous tumor cells and beads stimulating CD3 in combination with CD28.

5. CD83 is expressed on PBMC from a patient with colon carcinoma following stimulation with beads stimulating CD3 in combination with CD28.

6. A higher level of CD83 is expressed on PBMC from a patient with colon carcinoma following 2 days stimulation via CD3 plus CD28 than following stimulation via CD3 in combination with CD28 plus CD40.

7. Regression of K1735-500A2 melanoma cells which express anti-CD3 scFv, following transfection, when transplanted to immunocompetent syngeneic mice.

8. Regression of K1735-WT cells transplanted to syngeneic mice repeatedly immunized against K1735-500A2 cells.

9. Sequence of the anti-human CD3 scFv gene used for transfection.

10. Expression of anti-human CD3 scFv at the surface of two human cell lines following retroviral gene transduction.

11. Proliferation of human T cells cocultured with human cell lines expressing anti-CD3 scFv at their surface.

12. Resting human PBMC lyse cells from two human cell lines expressing anti-CD3 scFv at their surface.

13. K1735-500A2 cells, which express anti-CD3 scFv at their surface, inhibit tumor formation from admixed K1735-WT cells when the ratio between K1735-500A2 and WT cells is 1:10, demonstrating a “bystander effect”.

14. Splenocytes from mice immunized with K1735-500A2 cells proliferate when combined with irradiated K1735-WT cells but not when combined with Ag104 cells.

15. K1735-1D8 cells transplanted to syngeneic mice are rejected by a mechanism dependent on both CD4+ T cells and NK cells.

16. Immunization with K1735-1D8 cells, but not with irradiated K1735-WT cells, protects against outgrowth of transplanted K1735-WT cells by a mechanism that has memory and specificity.

17. Therapy of established K1735-WT tumors growing subcutaneously or in the lung using subcutaneously transplanted K1735-1D8 cells as a vaccine.

18. Splenocytes from K1735-1D8 immunized mice proliferate when combined with K1735-WT cells but not with cells from the antigenically different sarcoma Ag104.

19. IFN gamma secretion and CTL activity of spleen cells from mice immunized against K1735-1D8.

20. K1735-1D8 cells, which express anti4-1BB scFv at their surface, inhibit tumor formation from admixed K1735-WT cells when the ratio between 1D8 and WT cells is 1:10, demonstrating a “bystander effect”.

21. Sequence listing of the anti-human 4-1BB scFv (5B9).

22. TABLE 1.

23. TABLE 2.

24. TABLE 3.

25. TABLE 4.

### DESCRIPTION-MAIN EMBODIMENT

This invention describes methods and compositions useful for generating anti-tumor immunity. The first embodiment describes a novel method to obtain tumor-reactive T lymphocyte populations in vitro for therapeutic use in vivo by stimulating co-cultures of PBMC and tumor cells PBMC from cancer patients, including patients with advanced cancer (who are known from previous work to be immunosuppressed), with immobilized antibodies to CD3 in combination either with CD28 alone or with CD28 plus CD40. The second embodiment describes compositions comprising genes encoding anti-CD3 scFv or anti4-1BB scFv expressed at the cell surface and transfected cells expressing these genes for induction of anti-tumor immunity

Stimulation and activation of T cells with antibodies to CD3 and CD28 immobilized on magnetic beads is known to result in polyclonal T cell growth and production of multiple cytokines (Levine et al., J Immunol, 159, 5921-30, 1997; Garlie et al., J Immunother, 22, 336-45, 1999). However, a transition from polyclonal proliferation to the generation and or expansion of antigen-specific T cells after stimulation with antibodies to CD3 and CD28 has not been previously described. This was accomplished in the present invention by the addition of autologous tumor cells to the initial cultures. The tumor cells were destroyed within 48-72 hrs by the activated T cells, and monocytes in the cultures matured into CD83+ dendritic cells during the same time period as a result of exposure to lymphokines, including IFNγ and TNFα, secreted by the activated T cells. The dendritic cells take up killed tumor cells, and present tumor antigens to the activated T cells, promoting a continued proliferation and outgrowth of tumor specific T cells.

In another embodiment of this invention, we constructed genes encoding single chain antibody fragments (scFv) specific for CD3 and transfected them for expression at the surface of cells from human or mouse tumor lines. In both cases, such transfected cells could activate T lymphocytes which proliferated, formed lymphokines and killed the tumor cells. This was followed by experiments performed in vivo, showing that mouse tumor cells expressing anti-mouse CD3 at their surface are rejected by immunocompetent mice and induce systemic immunity capable of rejecting wild type cells from the same tumor. Thus, although scFvs encoded by the anti-mouse or anti-human CD3 genes induce polyclonal T cell activation when expressed on the tumor cell surface, our invention demonstrates that the polyclonal activation properties of anti-CD3 when tumor cells are present induces a transition to antigen-specific immunity when applied in vivo as a cancer vaccine.

In a third embodiment of this invention we constructed genes encoding single chain antibody fragments (scFv) specific for 4-1BB and transfected them for expression at the surface of tumor cells from humans and mice. Such transfected tumor cells can activate T lymphocytes from the respective species. Mouse tumor cells transfected with the anti-mouse 4-1BB scFv gene are rejected by immunocompetent mice and can be used as a vaccine to induce tumor specific immunity to wild type cells from the same tumor. The immune response, which we show has memory and is antigen specific, is therapeutically effective against the tumor cells studied (K1735 melanoma), growing subcutaneously or as lung metastases. These results are biologically significant since K1735 has very low immunogenicity, expresses very low levels of MHC class I molecules and lacks MHC class II and is thus similar to the majority of human tumors.

We have made genes encoding scFv molecules reactive with mouse and human CD3 and 4-1BB. Each gene contains the transmembrane domain and cyplasmic tail of human CD80. In addition, each gene encodes the hinge, CH2 and CH3 domains of human IgG1, located between the scFv binding site and the transmembrane domain. These genes and cells transfected with these genes are useful for therapy of cancer.

### EXAMPLE 1

### CD3-Mediated Activation of Tumor-Reactive Lymphocytes From Human Patients with Advanced Cancer

Peripheral blood mononuclear cells (PBMC) or tumor infiltrating lymphocytes (TIL) were cocultivated with autologous tumor cells in the presence of magnetic beads conjugated with a MAb to CD3 in combination with a MAb to CD28 or with MAbs to both CD28 and CD40. We characterize several things that occur in the cultures, including destruction of the cultured tumor cells, proliferation and lymphokine production of the lymphocytes, generation of CD83+ APC and activation/expansion of tumor-reactive CTL, as well as decreased sensitivity of the lymphocytes to inhibition by TGF-beta.

Patient material. Tumors were obtained at surgery or from malignant effusions (mostly ascites) of patients with stage IV carcinomas. Tumors and peripheral blood samples were provided by Dr Gary Goodman, Swedish Hospital Medical Center, under informed consent. Most studies were performed with 8 patients, 5 of whom (1OV, 3OV, 8OV, 44OV, 48OV) had ovarian carcinoma, 2 (1C, 22C) had colon carcinoma, and one (1HN) had a head and neck carcinoma. Cells from an ovarian carcinoma line, 4007, were also used.

Preparation of tumor and blood samples. Solid tumors were suspended in medium, and fluids were removed from effusions after which the cells were resuspended. Erythrocytes were removed by Ficoll-Hypaque (Pharmacia Biotech, Upsala, Sweden), and a Percoll gradient (Sigma, St Louis, Mo.) was used to separate tumor cells from TIL. Lymphocyte samples were used directly or stored in liquid nitrogen for later use. Tumor samples were explanted in vitro, using standard procedures, to establish cell cultures. PBMC containing T lymphocytes, monocytes and B cells, were purified using Ficoll-Hypaque. In a few initial experiments, CD8+ T lymphocytes (>90% pure) were used which had been positively selected from TIL using VarioMac magnetic beads (Miltenyi Biotech Inc., Auburn, Calif.).

Preparation of cultures combining lymphocytes, antibody-conjugated beads and tumor cells. In the initial experiments, 5 lymphocytes were added per tumor cell, after which the mixtures were incubated at 37° C. in Costar (3513) 12-well plates (Corning Inc., Corning, N.Y.) with RPMI medium (Gibco, Rockville, Md.) and 10% fetal calf serum (Atlanta Biological, Norcross, Ga.). They were followed by experiments in which PBMC or TIL were cultured with or without autologous tumor cells in the presence of magnetic beads (Dynal Inc., Lake Success, N.Y.) conjugated, using a published technique (Levine, Bernstein et al., J Immunol, 159, 5921-30, 1997; Garlie, LeFever et al., J Immunother, 22, 336-45, 1999), with MAbs to CD3, CD28, and/or CD40; beads not conjugated with MAb (or with an irrelevant MAb) were used as controls. The MAbs were 64. 1(Martin et al., J Immunol, 136, 3282-7, 1986) (Martin, Ledbetter et al., J Immunol, 136, 3282-7, 1986), 9.3 (Martin, Ledbetter et al., J Immunol, 136, 3282-7, 1986)and G28-5 (Ledbetter et al., J Immunol, 138, 788-94, 1987), which, respectively, stimulate lymphocytes polyclonally (anti-CD3), costimulate them (anti-CD28), or activate APC (anti-CD40). When autologous tumor cells were used, cells (40,000-75,000/well) were first attached by overnight incubation to Costar 24-well plates containing 2 ml IMDM medium with 10% fetal bovine serum. MAb-conjugated beads (3×10⁶/ml) were then added, followed by lymphocytes (10⁶/ml) in RPMI with 10% fetal bovine serum. The plates were incubated at 37° C. in a 6% CO₂ in air atmosphere for 4-5 days. The beads were then removed using a magnet, and the lymphocytes placed in new wells in medium containing 10 U/ml of IL2 (Roche Molecular Biochemicals, Indianapolis, Ind.) and moved into flasks when their concentration had reached 2×10⁶ cells/ml. Cultures were observed for evidence of tumor cell destruction. Lymphocyte proliferation was determined by cell counting. Media were sampled to measure production of TNF in a bioassay using WEHI cells (Espevik and Nissen-Meyer, J Immunol Methods, 95, 99-105, 1986) and IFN-gamma was measured by an ELISA (EH-IFNG, Endogen, Woburn, Mass.), respectively. TGFβ1 was purchased from Sigma (St Louis, Mo.). In all experiments using TGFβ1, the molecule remained in the cultures, also after removal of MAb-conjugated beads.

CTL assays. Classical 4-hour ⁵¹Cr release assays were performed. To characterize the effector cells, experiments were done to inhibit cytotoxicity by addition of MAb w6/32 (10 ug/ml) which recognizes a MHC class I frame-work determinant (Research Diagnostics Inc., Flanders, N.J.). MAbs to the NK markers CD16 and CD56 (Beckman Coulter, Brea, Calif.), anti-CD8 MAb HIT8a (BD Pharmingen, Lexington, Ky.), and anti-integrin-beta 2 (CD18) MAb 60.3 (Beatty et al., J Immunol, 131, 2913-8, 1983) were also used.

FACS analysis of lymphocytes. Density of CD expression was evaluated by FACS (Epics XL, Coulter, Miami, Fla.), using PE-labeled MAb and counting cells as positive when they had a pre-set minimum brightness. To investigate whether an increased density of CD3 expression after in vitro activation of lymphocytes was due to the selective proliferation of cells with originally high CD3 expression, PBL harvested from cancer patients were labeled with the dye CFDA (den Haan et al., J. Exp. Med., 192, 1685-1695, 2000) (Molecular Probes, Eugene, Oreg.). Subsequently, they were cultured in the presence of anti-CD3/CD28/CD40 beads for 5 days, after which the beads were removed and the lymphocytes expanded in medium containing 10 U IL-2/ml. At two time points after removal of the beads (4 hours and 3 days) FACS analysis was performed, in which cells were analyzed for CFDA brightness and for expression of CD3. Labeled lymphocytes which had been cultured with control beads were studied for comparison.

Demonstration of low levels of T cell reactivity in the absence of stimulation via antibody-conjugated beads. Six initial experiments were performed in which CD8+ T lymphocytes purified from TIL were cultured with tumor cells, after which the supernatants were assayed for TNF or IFN-gamma. In a representative experiment, CD8+ TIL from a colon cancer patient, 1C, first cultivated with 1C tumor cells for 15 days, were removed and added to either a fresh set of 1C cells or to tumor cells from a lung carcinoma patient, 3L. A small amount of TNF (1.2 pg/ml) was detected when 1C lymphocytes were combined with the 1C but not with the 3L tumor, while TNF and IFN-gamma (1.5 pg/ml) were produced when TIL from 3L were combined with 3L tumor cells but not when cultured alone. There was no evidence of lymphocyte proliferation. In subsequent experiments, TIL populations comprising monocytes, CD4+ T cells and B cells in addition to CD8+ lymphocytes were combined with autologous tumor cells and cultured for 10-15 days. Approximately 10 times higher levels of TNF (4.5-48 pg/ml) and IFN-gamma (up to 150 pg/ml) were then detected in supernatants from cultures of 8 of 13 patients. There was still no lymphocyte proliferation.

Demonstration of T cell proliferation and tumor cell destruction in the presence of autologous tumor cells and anti-CD3-conjugated beads. The initial experiments were followed by experiments in a system in which MAb-conjugated magnetic beads are used to induce signals via various lymphocyte receptors (Levine, Bernstein et al., J Immunol, 159, 5921-30, 1997; Garlie, LeFever et al., J Immunother, 22, 336-45, 1999). PBMC or TIL were combined with autologous tumor cells in the presence of beads conjugated with MAbs to CD3 and MAbs to CD28, alone or together with CD40. Similar groups were included with lymphocytes but without tumor cells. As controls, lymphocytes, with or without tumor cells, were cultivated with control, unconjugated beads. Following 3-5 days, the beads were removed and the lymphocytes and tumor cells incubated separately over a 2-21 day period with 10 U/ml of IL2.

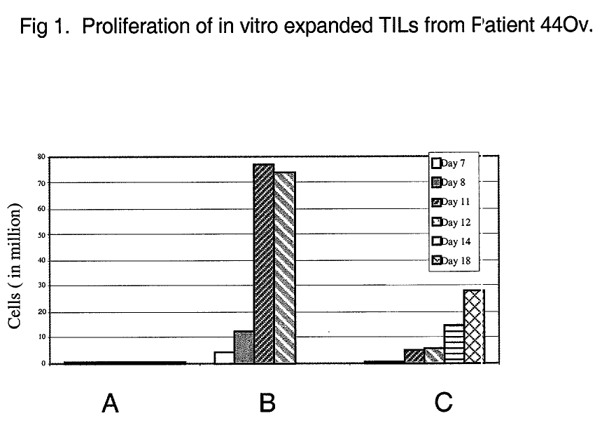

FIG. 1 shows an experiment in which TIL from patient OV44 proliferated vigorously when exposed for 4 days to anti-CD3/CD28/CD40 conjugated beads. Lymphocytes cultivated in the absence of a CD3 signal did not proliferate and neither did lymphocytes cultured with anti-CD28 and/or CD40 beads (data not shown). Proliferation was greater when autologous tumor cells were initially present with the beads inducing signals via CD3 (panel B). Anti-CD3/CD28 conjugated beads induced proliferation similar to that with anti-CD3/CD28/CD40 conjugated beads (data not shown).

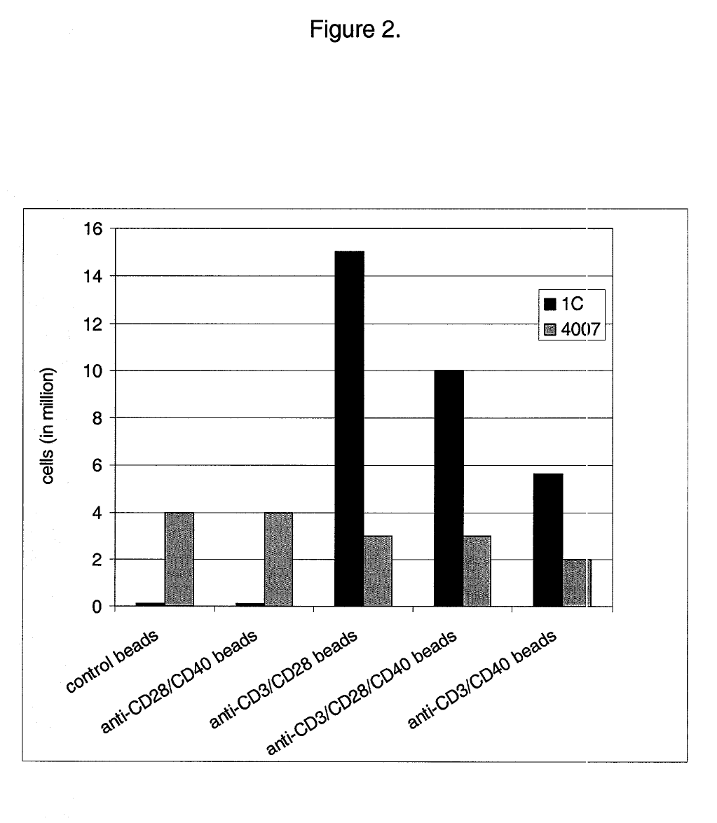

FIG. 2 shows an experiment in which PBL from patient 1C and various MAb-conjugated beads were cultivated for 5 days with either autologous tumor cells or allogeneic (4007) cells. The number of lymphocytes per culture was much higher when CD3/CD28 (panel C) or anti-CD3/CD28/CD40 (FIG. 2D) activated lymphocytes were combined with 1C tumor than with 4007 cells, a finding similar to that illustrated in FIG. 1. FACS analysis showed that >90% of the activated lymphocytes expressed CD3 and a approximately 70% of them were CD8+, with less than 5% expressing CD16 or CD56. When, on the other hand, the beads did not provide any signal via CD3 (FIG. 2A and B), the proliferation was higher when allogeneic cells were added, and probably represented an immunological response to alloantigens expressed on the 4007 cells.

Most of the tumor cells were destroyed within 24-48 hours after exposure to autologous lymphocytes in the presence of anti-CD3/CD28 or anti-CD3/CD28/CD40 conjugated beads, often leaving cultures entirely comprising cells with lymphocyte morphology. In order to study whether this tumor destruction had immunological specificity, 4 experiments were performed in which serial dilution of PBL (10⁶-10⁵/sample) from cancer patients were combined with autologous tumor cells or with either tumor cells or fibroblasts from an allogeneic donor. In both of 2 experiments, there was approximately 10 times more TNF in the culture supernatants in the presence of the autologous tumor, but there was no difference in the killing of cells from autologous or allogeneic tumors or of allogeneic fibroblasts. We conclude that tumor cell destruction seen after 24-72 hours in the presence of lymphocyte activation was not antigen specific, perhaps because large amounts of activated T lymphocytes and lymphokines obscured any specific components.

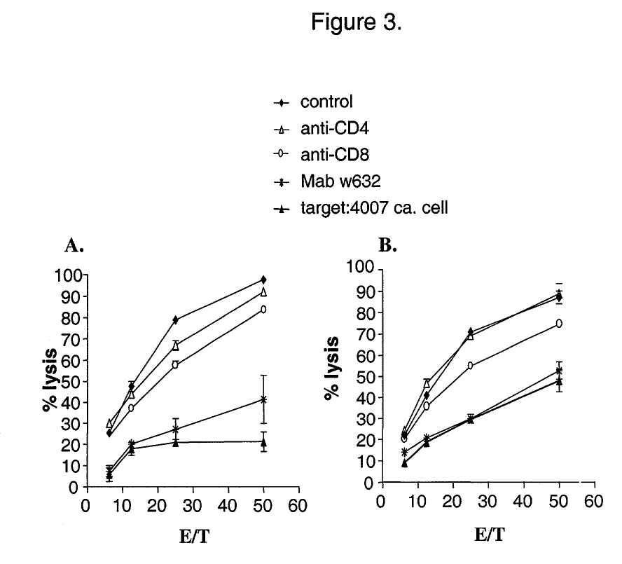

Generation of tumor-selective CTL. MHC-class I-restricted CTL were generated from lymphocytes activated by tumor cells plus anti-CD3/CD28 or anti-CD3/CD28/CD40 beads. FIG. 3 presents an experiment with PBL from patient 1C, which had been activated in the experiment shown in FIG. 2. After activation by tumor cells and MAb-conjugated beads, the beads were removed and the lymphocytes expanded with 10 U IL-2/ml medium over 3 weeks in the absence of additional tumor cells and beads. PBL activated by 1C and anti-CD3/CD28 beads were strongly cytolytic to 1C cells, and lysis was inhibited by a MAb to CD8 and by anti-MHC Class I framework MAb w6/32 (FIG. 3A). Allogeneic 4007 cells were killed by only 20% at an E/T of 50:1, as compared to 98% lysis of 1C cells (FIG. 3A). FIG. 3B demonstrates analogous data for PBL stimulated with anti-CD3/CD28/CD40 beads. Lysis of 4007 cells was then at the same low level as that of 1C in the presence of MAb w6/32. In contrast, PBL stimulated with anti-CD3/CD28/CD40 beads killed both 1C and 4007 cells, also in the presence of MAbs to CD8 or MAb w6/32 (data not shown). CD8+ cells enriched from the cell population used in the experiment shown in FIG. 3B lysed 25% of 1C cells at an E/T ratio of 20/1 as compared to 0% of cells from the 4007 line and 0% of cells from an allogeneic B cell line. In this experiment, lysis of 1C cells was 5% in the presence of MAb w6/32 and 5% with the anti-CD18 MAb 60.3, and it only decreased from 25% to 18% with a combination of MAbs to CD16 and-CD56. Lymphocytes activated by cocultivation with 4007 cells and any of the beads did not selectively lyse 1C or 4007 cells. The CTL assays were repeated twice with similar results.

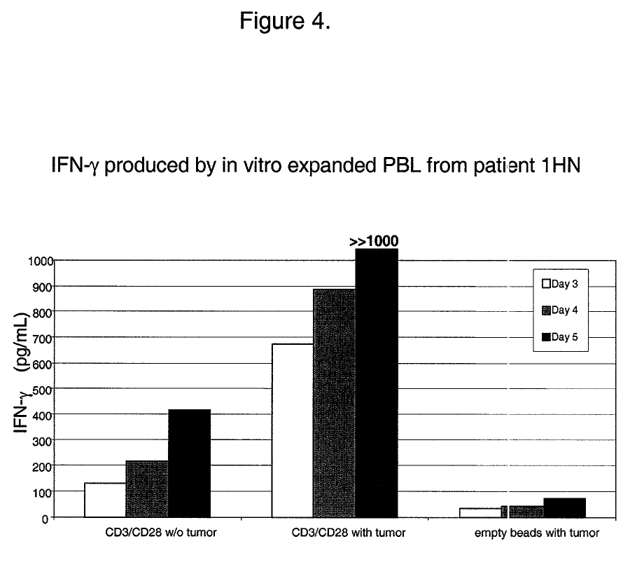

Production of Th1 type lymphokines. Large amounts of IFN-gamma were detected in supernatants of cultures from lymphocytes activated via CD3. This is illustrated in FIG. 4, which also shows that the production of IFN-gamma was higher when autologous tumor cells were present during the first 4-5 days of culture.

Table 1 presents 6 additional, representative experiments showing proliferation and lymphokine production by PBL or TIL which were either tested upon harvest from the patients or after one round of in vitro activation with beads. Anti-CD3, anti-CD3/CD28, anti-CD3/CD40 and anti-CD3/CD28/CD40 beads strongly increased lymphocyte proliferation with no significant difference between them. In contrast, anti-CD28, anti-CD40 and anti-CD28/CD40 beads alone did not increase lymphocyte proliferation and lymphokine production over control beads, indicating that signaling via CD3 was essential. Production of TNF and IFN-gamma correlated with each other. It decreased to background levels when the lymphocytes were grown without tumor cells and beads for more than 3-5 days. As in FIGS. 1 and 4, CD3 signaling was required to induce vigorous lymphocyte proliferation and lymphokine production.

Upregulation of CD3 and other markers on lymphocytes activated via MAb-conjugated beads. The density of CD antigen expression on lymphocyte populations was measured by FACS before and after 3 to 5 day cultivation with tumor cells and anti-CD3/CD28/CD40 beads, followed by an additional 3 to 7-day expansion without beads. To reflect changes in the density of CD receptor expression, the percentage of cells in each population whose brightness equaled the density at the chosen setting, or was higher, is reported (Table 2); unstimulated PBL from 6 healthy donors (30 to 65 years of age) were analyzed for comparison. Unstimulated PBL from the cancer patients had low levels of CD3, CD4 and CD28. Four of 5 patients also had low CD8 density, while the CD86 density was higher than among unstimulated PBL from the healthy donors. Culturing of PBL with control beads partially increased CD3 expression, but did not significantly increase CD28 expression. In contrast, culturing with anti-CD3/CD28/CD40 beads consistently restored the expression of CD3 and CD28 to normal levels, and it doubled the number of cells with high density CD8 expression. Density of CD3 expression was studied with TIL from 5 patients. It was 2.9%, 40.2%, 96%, 42.8% and 40.1%, respectively, i.e. it displayed more variation and was generally higher than for PBMC. CD8 expression by TIL was higher than among PBMC and increased from 61.4% to 87.3%. The corresponding figures for CD28 expression among TIL were 39.3% and 52.8%.

To investigate whether an increased density of CD3 expression after in vitro activation of lymphocytes was due to the selective proliferation of cells with originally high CD3 expression, PBL harvested from cancer patients were labeled with the dye CFDA (Molecular Probes, Eugene, Oreg.). Experiments were performed with TIL, 40.2% of which originally expressed CD3, were labeled with the dye CFDA (den Haan, Lehar et al., J. Exp. Med., 192, 1685-1695, 2000). After activation via anti-CD3/CD28/CD40 beads, CD3 expression increased to 95%. FACS analyses, using CFDA and PE-labeled anti-CD3 as probes, showed that there was no selective proliferation of the subpopulation of PBL that originally had higher CD3 expression.

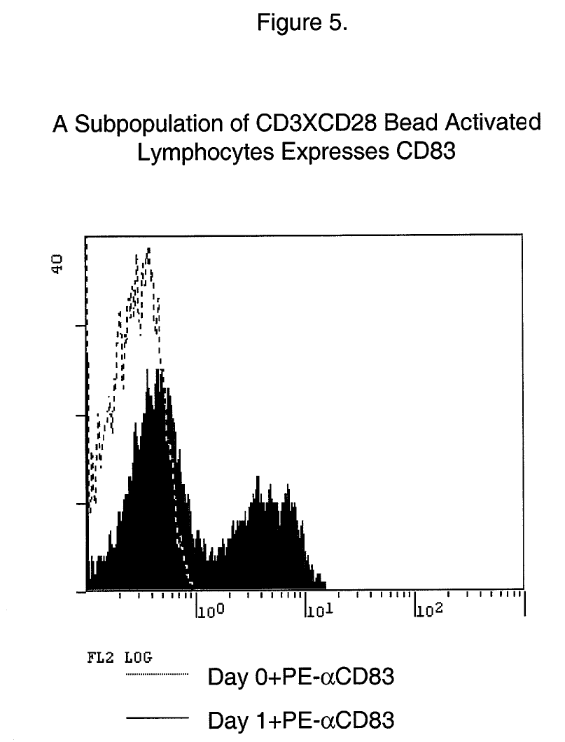

Expression of CD83 in cultures after stimulation with anti-CD3/CD28 beads. To investigate whether stimulation of PBMC, of which 10-20% were found to express the monocyte marker CD14, with anti-CD3/CD28/CD40 beads could increase the maturation of dendritic cells, we measured the expression of CD83 at various times after stimulation. CD83 is expressed by dendritic cells after maturation but is not expressed by immature dendritic cells or blood monocytes. FIG. 5, which illustrates a typical experiment, shows that as early as 24 hrs after stimulation of PBMC with anti-CD3/CD28 beads, expression of CD83 was detected on 35% of the cells. No expression of CD83 could be detected on day 0 PBMC (before stimulation).

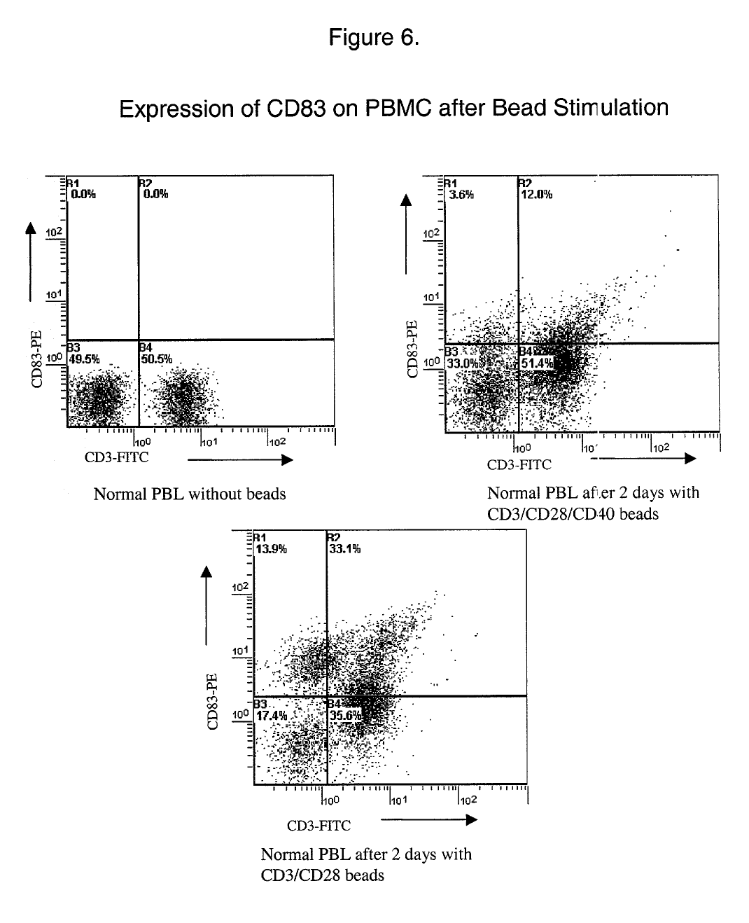

To determine what cells express CD83 after PBMC stimulation, two color staining was performed with fluorescein labeled anti-CD3 versus PE-labeled anti-CD83 on day 2 following stimulation with anti-CD3/CD28 beads or anti-CD3/CD28/CD40 beads. FIG. 6 shows that while CD83 was not expressed on cells in the absence of bead activation, the beads conjugated with anti-CD3/CD28 induced CD83 expression on a distinct population of CD3 negative cells (13.9%), and also on a significant proportion of CD3 positive cells. In contrast, activation with beads conjugated with anti-CD3/CD28/CD40 induced expression of CD83, but to a lower level than the anti-CD3/CD28 beads on both CD3 negative and CD3 positive cells. These results show that cells that express the dendritic cell marker CD83 are rapidly induced from PBMC after stimulation with beads conjugated with anti-CD3 and anti-CD28 MAbs. Stimulation with beads conjugated with anti-CD3, anti-CD28, and anti-CD40 MAbs were not as effective as beads conjugated with anti-CD3 and anti-CD28 MAbs alone in stimulation of CD83 expression.

Increased resistance to inhibition by TGFβ1 in the presence of activation signals via MAb-conjugated beads. Table 3 shows 5 representative experiments performed to investigate whether the inhibitory effect of TGFβ1 on lymphokine production and lymphocyte proliferation could be altered by co-culture with beads inducing signals via CD3. With control beads, the TNF and IFN-γ levels were low, and these levels were further suppressed by TGFβ1. In contrast, with anti-CD3/CD28/CD40 beads these levels increased to levels approaching those seen in the absence of TGFβ1. Likewise, when anti-CD3/CD28/CD40 beads were used, there was much less inhibitory effect of TGFβ1 on lymphocyte proliferation with no inhibition at all seen with patient 1HN. A relative resistance of T cell proliferation and lymphokine production was seen also when the TGF-beta 1 dose was increased to 20 ng/ml and when the concentration of lymphocytes was decreased to 10⁵/sample (data not shown). Beads stimulating via CD28, CD40, alone or together, did not protect against TGFβ1 (data not shown).

Conclusions. Lymphocyte activation in the presence of tumor cells, accompanied by tumor cell killing, causes the release of antigen. Monocytes in the cultures take up tumor antigen, differentiate into CD83 positive APC, and present epitopes for the selective expansion of tumor-reactive T cells. Therapeutic vaccines can be based on the same principle to activate and expand suppressed lymphocytes in tumor-bearing individuals and may also facilitate the generation of immune responses to subdominant epitopes. The culture system for generation of tumor reactive T cells includes four components. These are

1) T cells from a patient with cancer,

2) antigen presenting cells from the same patient,

3) beads conjugated with anti-CD3 and anti-CD28 antibodies or with anti-CD3, anti-CD28 and anti-CD40 antibodies, and

4) tumor cells from the same patient.

There are many variations of these components that are envisioned. These include variations in the time of addition of any of the four components, as well as variations in the origins of the components. For example, patient T cells can be isolated from peripheral blood or from tumor infiltrating lymphocytes. Antigen presenting cells, in the examples shown were present in the peripheral blood mononuclear cell fraction, but can also be derived from other sources such as bone marrow. While the examples shown used autologous tumor cells in the culture, allogeneic tumor cells or tumor antigens could also be used in addition to or instead of autologous tumor cells, since the tumor antigens are presented by autologous APC. Magnetic beads conjugated with anti-CD3 and anti-CD28 antibodies can be replaced with antibodies immobilized in other ways, and can be composed of immobilized antibodies or ligands specific for additional cell surface receptors that promote polyclonal T cell activation and expansion of tumor reactive T cells.

The procedures we have used make possible the generation of CD3 positive lymphocytes, which continue to expand over >10 weeks of in vitro culturing and are useful for adoptive immunotherapy. This may be because costimulation via CD28 decreases the probability for lymphocytes to undergo apoptosis (Boise et al., Immunity, 3, 87-98, 1995; Daniel et al., J Immunol, 159, 3808-15, 1997), providing them with a long life span in vitro (Levine, Bernstein et al., J Immunol, 159, 5921-30, 1997). Costimulated lymphocytes have also survived for a long time following transfer back to autologous patients (Ranga et al., Proc. Natl. Acad. Sci., 95, 1201-1206, 1998) as opposed to lymphocytes expanded in the presence of high doses of IL2. It is noteworthy that T cell stimulation via CD3 in combination with CD28 alone or together with CD40 can protect against approximately 50% of a TGFβ1-mediated inhibitory effect on lymphocyte proliferation and production of TNF and IFN-gamma, even when the TGFβ1 was used at saturation levels of 20 ng/ml in the cultures.

### EXAMPLE 2

### Construction of Vectors Encoding Anti-human and Anti-mouse CD3 scFv of Human or Mouse Origin, Transfection, and Demonstration that Cells Expressing Anti-CD3 scFv at Their Surface Induce Polyclonal Stimulation of T Cells to Proliferate, Produce Th1 Type Lymphokines and Become Cytolytic and to Have Anti-tumor Activity in vivo

The experiments described above show that a signal provided by anti-CD3 mAb conjugated to the magnetic beads was necessary for the activation and expansion of tumor reactive lymphocytes in the cultures containing autologous tumor cells plus PBMC or TIL. We therefore constructed genes for tumor therapy that allow expression of active anti-CD3 mAb single chain Fv (scFv) derivatives at the tumor cell surface. Anti-CD3 scFv reactive with mouse CD3 was constructed from hybridoma 500A2, provided by Dr J. Allison, University of California, Berkeley, Calif., and anti-CD3 reactive with human CD3 was constructed from hybridoma G19-4 (Ledbetter et al., J.Immunol., 136, 3945-3952, 1986).

Construction of scFvs. Cell surface forms of single chain Fv (scFvs) were constructed by cloning the variable domains for the light and heavy chains of the antibodies from the hybridoma RNA (Hayden et al., Ther Immunol, 1, 3-15, 1994; Gilliland et al., Tissue Antigens, 47, 1-20, 1996). Hybridomas were grown in RPMI containing [10% fetal bovine serum, 4 mM glutamine, 1 mM sodium pyruvate, and 50 u/ml penicillin-streptomycin, (all from Life Technologies, Gaithersburg Md.)] and maintained in logarithmic growth for several days prior to cell harvest. Cells were harvested by centrifugation from the suspension cultures, and RNA isolated from 2×10⁷ cells by Trizol or using QIAGEN RNA columns (Life Technologies, Gaithersburg Md., and QIAGEN, Valencia, Calif.) according to the manufacturer's instructions or by a modified version of the NP-40 Lysis technique (Gilliland, Norris et al., Tissue Antigens, 47, 1-20, 1996). One microgram of total RNA was used for random primed first strand synthesis of cDNA using Superscript II Reverse Transcriptase (Life Technologies) and random hexamers (Takara Shuzo, Otsu Shiga, Japan). Following reverse transcription, cDNA fragments are poly G-tailed using dGTP and terminal transferase, an enzyme that catalyzes the addition of deoxyribonucleotide from deoxynucleotide triphosphates to the terminal 3′-OH group of a DNA strand. cDNA was anchor tailed in order to increase the efficiency of cloning mRNA with unknown leader peptides at one end. The 5′ primer is a modified ANCTAIL primer containing a poly C tail as described for PCR of T cell receptor chain sequences (Loh et al., Science, 243, 217-20, 1989), but with SacI, XbaI, and EcoRI sites for cloning purposes. The sequence is as follows:

5′-cgtcgatgagctctagaattcgcatgtgcaagtccgatgagtccccccccccccc-3′

Primers for the 3′ end of the cDNA were from the constant region of the heavy or light chain, and bind approximately 50 bases beyond the J-C junction. Each 3′ primer contained HindIII, BamHI, and Sal I sites for cloning. Restriction sites for subcloning the initial fragments were thereby incorporated as part of these original PCR amplification primers, and amplified PCR fragments were digested and subcloned into pUC19, pSL1180, or into TOPO vectors (Invitrogen, San Diego, Calif.) for sequencing. DNA sequencing was performed on miniprep DNA (QIAGEN, Valencia, Calif.) using pUC, T7, or M13 universal and reverse primers and BigDye Terminator Cycle Sequencing Kit Reagents (PE Biosystems, Foster City, Calif.) on an ABI Prism 310 (PE Biosystems) Sequencer.

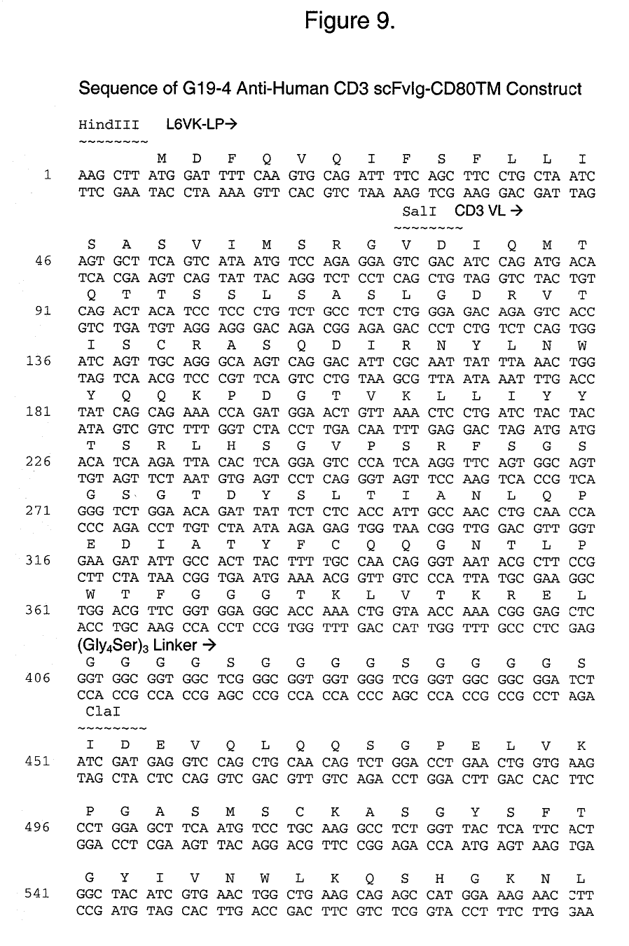

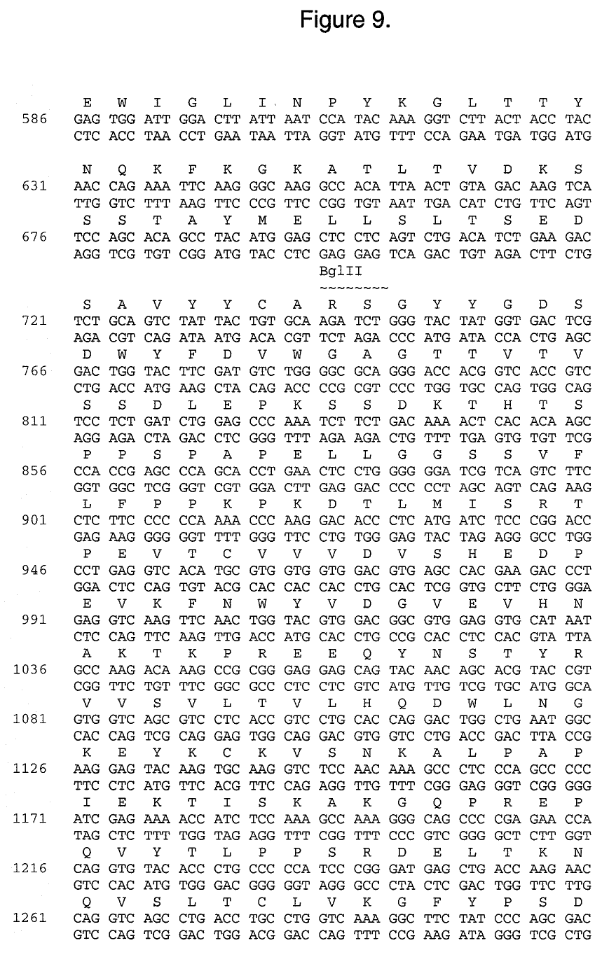

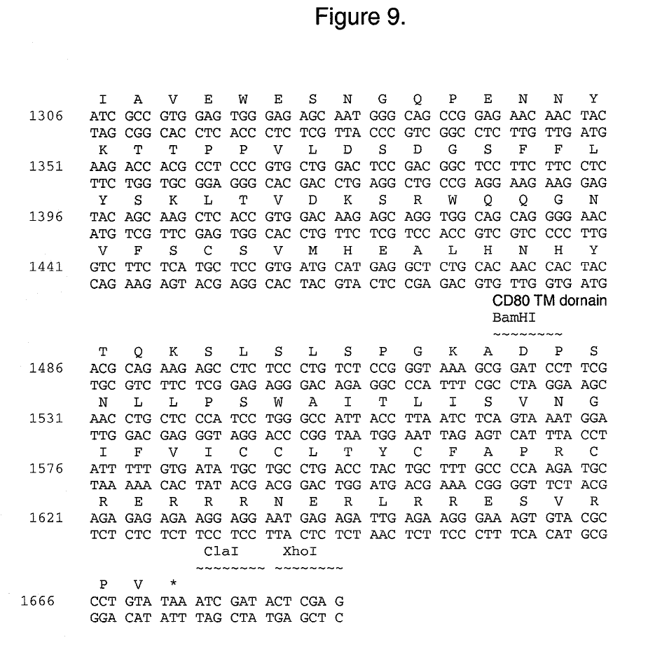

Once individual variable domains were isolated and consensus sequence generated from at least three identical clones, the scFv was constructed by PCR amplification using overlapping oligonucleotides that result in the fusion of cDNAs encoding the light and heavy chain variable regions. Light and heavy chain variable domains were connected during this sewing PCR by the addition of a (gly₄ser)₃ linker as part of the overlapping oligonucleotides (Gilliland, Norris et al., Tissue Antigens, 47, 1-20, 1996). The assembled scFv molecules were subcloned upstream of the human IgG1 hinge, CH2, and CH3 domains fused in frame to the human CD80 transmembrane and cytoplasmic tails (Winberg et al., Immunol Rev, 153, 1996). Completed expression cassettes encoded either the native leader peptide for the light chain V region or the secretory signal peptide from the L6 VK light chain fused at a SalI site to the light chain variable region of the scFv. The scFv was encoded as a HindIII-BclI, or SalI-BclI cassette, where the first restriction site was encoded in frame with respect to the open reading frame, while the second restriction site was out of frame with respect to the reading frame for the fusion protein. This cassette was then fused to the human IgG1 wild type Fc domain encoded on a BglII-BamHI fragment. The CD80 transmembrane and cytoplasmic tails were amplified by PCR from human tonsil RNA and encoded on a BstBI-ClaI fragment including a STOP codon just upstream of the ClaI site. Each subfragment was subcloned into a synthetic polylinker/multiple cloning site that had been inserted into a modified version of the vector pCDNA3. Once the complete fusion protein construct encoding the cell surface scFv was assembled, the entire expression cassette was transferred to the retroviral expression vector pLNCX (Miller and Rosman, Biotechniques, 7, 980-2, 984-6, 989-90, 1989) as a HindIII-ClaI fragment (FIG. 9).

Transfections. Plasmid DNA was prepared from these recombinant retroviral vectors, and used to transfect PT67 dual tropic (Clontech, Palo Alto, Calif.) packaging cells by the CaPO₄ precipitation technique (Winberg, et al., (1996) Immunol Rev. 153: 209-223.). Briefly, cells were plated at approximately 25% confluency in DMEM containing 10% fetal bovine serum, 4 mM glutamine, 2×DMEM non-essential amino acids, and penicillin-streptomycin (this formulation is subsequently referred to as DMEM-C and all reagents are from Life Technologies) and grown overnight prior to transfection. Plasmid DNA was added to 0.5 ml 0.25 M CaCl₂ and then added dropwise to 0.5 ml 2×HEBS buffer (pH 7.1). Precipitates were allowed to form for 5 minutes at 37° C., and the solutions were then added dropwise to cells in 100 mm culture dishes containing fresh DMEM-C (8 ml). Transfected cells were incubated overnight and then washed twice in PBS and fed with fresh media. Viral supernatants were harvested from transfected cells and used for transduction 24 hours later. Alternatively, transfected, adherent PT67 cells expressing the cell surface scFv were co-cultured with the B cell lines growing in suspension. After several passages, the packaging cells were diluted from the culture and the B cell lines could be panned for expression of the cell-surface scFv using goat anti-human IgG1 immobilized on culture flasks. Cells expressing high levels of the cell surface scFv bound more tightly to the flask and negative cells and low expressers were washed from the flask. High-level expressers could then be isolated by scraping them from the flask surface and reculturing for a few days prior to use in biological assays.

Mice and tumor cell lines. Six to eight-week old female C3H/HeN mice were purchased (Taconic, Germantown, N.Y.). K1735 is a melanoma of C3H/HeN origin from which a metastatic clone, M2, was selected.(Fidler and Hart, Cancer Res, 41, 3266-3267, 1981) In agreement with previous findings, its MHC class I expression was found to be very low (data not shown). The animal facilities are ALAC approved and our protocols were approved by PNRI's Animal Committee.

Antibodies. R-phycoerythrin (PE)-conjugated MAbs GK1.5 (anti-mouse CD4), 53-6.7 (anti-mouse CD8a) and purified AF3-12.1(anti H-2K^{K}) were from Pharmingen (San Diego, Calif.) and R-PE conjugated goat F(ab′)₂ anti-human IgG from Biosource International (Camarillo, Calif.). MAbs 169-4 (anti-CD8) was from Dr R. Mittler (Emory University, Atlanta, Ga.). GK1.5 (anti-CD4) was produced by a hybridoma obtained from ATCC.

Vectors and transfection of K1735 cells. Methods of variable region cloning, scFv construction, and generation of scFv expression have been described (Gilliland, Norris et al., Tissue Antigens, 47, 1-20, 1996; Hayden et al., Tissue Antigens, 48, 242-54, 1996; Winberg, Grosmaire et al., Immunol Rev, 153, 1996). The present studies were performed with variable region genes from the anti-CD3 hybridoma 500A2 or the anti-4-1BB hybridoma 1D8, provided by Dr J. P. Allison, University of California, Berkeley, Calif. and Dr. R. Mittler, Emory University, Atlanta, Ga.) to obtain surface expression of cell-bound 500A2 scFv or 1D8 scFv (Hayden, Grosmaire et al., Tissue Antigens, 48, 242-54, 1996; Winberg, Grosmaire et al., Immunol Rev, 153, 1996). For expression of scFv, the transmembrane domain and cytoplasmic tail from CD80 was used, since it mediates cytoskeletal attachment and crosslinking during cell-cell contact (Doty and Clark, J Immunol, 157, 3270-9, 1996; Doty and Clark, J Immunol, 161, 2700-7, 1998). The scFv gene fusion construct in pLNCX was transfected into RetroPack™ PT67 packaging cells (Clontech Laboratories, Inc, Palo Alto, Calif.) by CaPO₄ precipitation. K1735-WT cells were transfected using medium from those cells. G418 resistant clones were stained by PE labeled goat anti-human IgG for scFv surface expression.

Animal Studies. Mice, 5 or 10/group, were transplanted s.c. on one side of the back with 2×10⁶ K1735-WT or K1735-500A2 cells or with gamma-irradiated (12,000 rads) K1735-WT cells. Immunized mice were challenged with K1735-WT (2×10⁶ cells/mouse) or Ag104 (3×10⁵ cells/mouse). Tumor size was assessed by measuring the two largest perpendicular diameters with calipers and reported as average tumor area (mm²)±SD. Sites where mice were transplanted s.c. were shaved to facilitate tumor measurements.

In Vivo Depletion of CD4⁺ and/or CD8⁺ T lymphocytes and of NK cells. T cells were depleted as described (Chen, Ashe et al., Cell, 71, 1093-102, 1992), injecting mice i.p. 3 times with MAb to CD4 (GK1.5, rat IgG2b) or CD8 (1694, rat IgG 2a), or with a mixture of the two, at 0.5mg/mouse for 3 consecutive days. This was followed by 0.5 mg of each MAb every 3 days to maintain the depletion. NK cells were depleted by injections of anti-asialo GMI antibodies at 30 μl/mouse i.p. every 4 days. On day 12, spleen cells from each group were analyzed by FACS to verify the efficiency of the depletions. Subsequently, the mice were transplanted s.c. with tumor cells.

Proliferation Assays. Spleen cells were seeded into 96-well flat-bottom plates (1×10⁵ cells/well) together with 5×10⁵ syngeneic, irradiated (3,000 rads) spleen cells (as APC) and tumor cells. After incubation for 72 hours, triplicate cultures were pulsed for 16-18 h with 1 μCi ³[H] thymidine (Amersham Pharmacia Biotech Piscataway, N.J.), the uptake of which was measured.

To investigate which T cells proliferated in vitro, spleen cells were labeled by incubation with 2μM CFDA SE (5-(and-6)-Carboxyfluorescein diacetate succinimidyl ester) (den Haan, Lehar et al., J. Exp. Med., 192, 1685-1695, 2000) according to the manufacturer's (Molecular Probes, Eugene, Oreg.) protocol, incubated with or without K1735-WT cells for 3 days and analyzed by FACS.

Assay for CTL Activity. Mice were sacrificed 2-4 weeks after transplantation of K1735-1D8, and spleen cell suspensions prepared. When stated, NK cells were removed using anti-asialo GM 1 antibodies plus rabbit complement (Cedarane, Ontario,Canada). 5×10⁶ splenocytes were cultivated for 5 days with 1×10⁵ □-irradiated (12,000 rads) K1735-WT cells in a 24-well plate (Costar Corp., Cambridge, Mass.). Cytolytic activity was examined in a 4-h Cr⁵¹ release assay at different E/T ratios.

ELISPOT assays. Murine IFNγ ELISPOT kits (R&D Systems, Minneapolis, Minn.) were used according to the manufacturer's protocol, and the plates were counted by Plate-scanning service (Cellular Technology Ltd., Cleveland, Ohio).

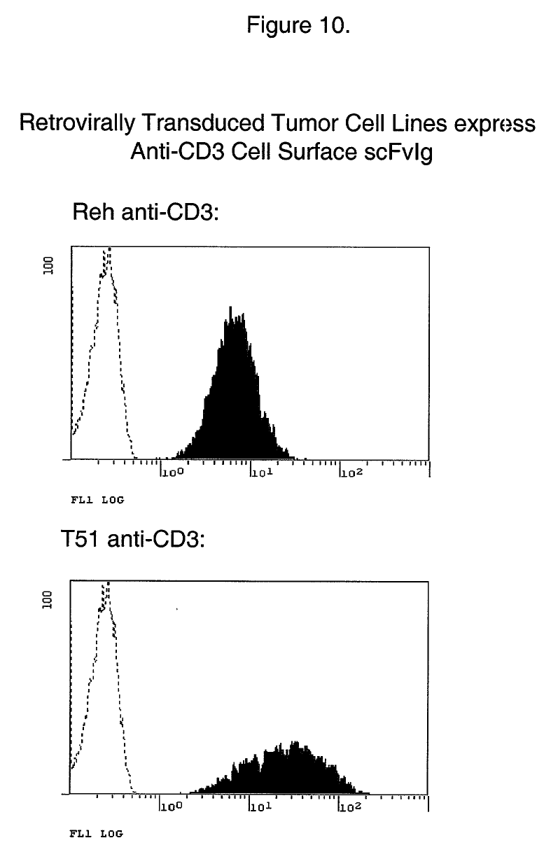

Polyclonally activated human T cells proliferate, produce Th1 type lymphokines and become cytolytic. Expression of the anti-human CD3 scFv at the cell surface of Reh, a reticuloendothelial 1 cell line, and T51, a B cell lymphoblastoid line is shown in FIG. 10. The transfected cells showed high levels of expression of the anti-CD3 scFv gene product.

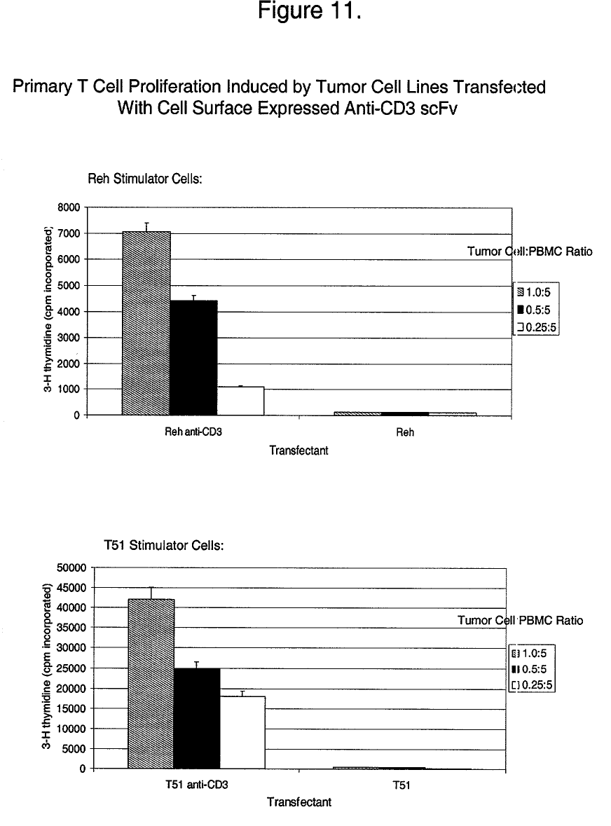

The ability of transfected versus wild type Reh and T51 cells to induce proliferation of T cells was tested by culture of the cell lines with PBMC from a normal donor. The wild type and transfected cell lines were treated with mitomycin C to prevent their proliferation during the culture. The transfected Reh and T51 cells expressing anti-CD3 scFv induced proliferation in a dose-dependent manner, while the wild type Reh and T51 cells did not (FIG. 11).

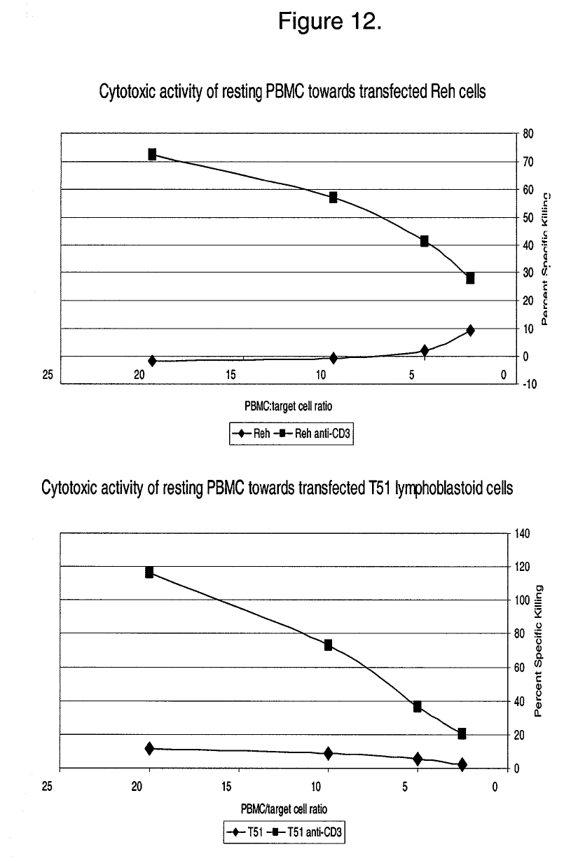

Experiments were performed to test whether the expression of anti-human CD3 scFv at the tumor cell surface stimulated T cells from PBMC to rapidly kill the transfected cells. Wild type or transfected Reh and T51 cells were labeled with ⁵¹Cr, and an 8 hr ⁵¹Cr release assay was performed using PBMC from a normal donor. FIG. 12 shows that the transfected but not the wild type cells were rapidly killed by resting T cells, resulting in significant release of ⁵¹Cr in a dose-dependent manner.

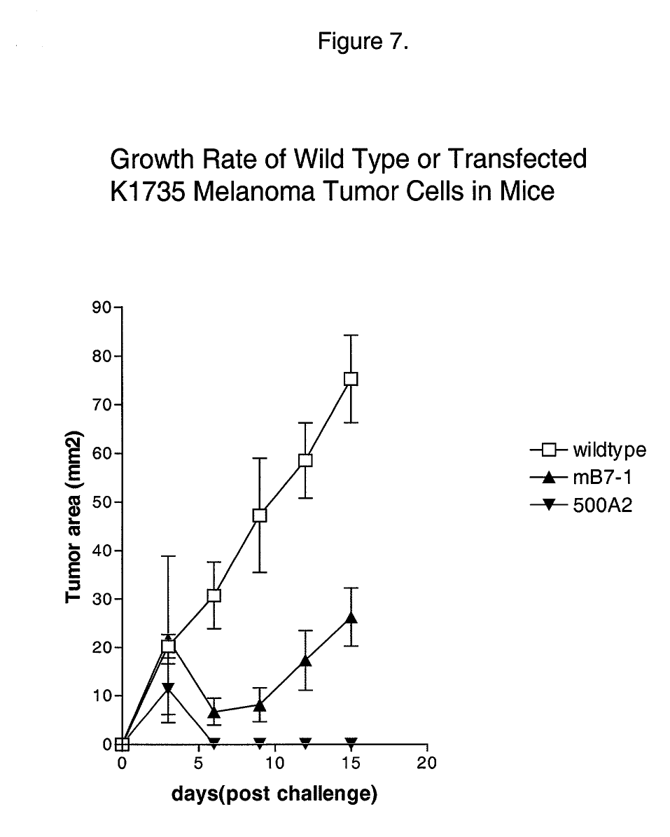

K1735-500A2 cells are rejected by immunocompetent syngeneic mice. As seen in FIG. 7, K1735-500A2 cells, which had been transfected to express anti-mouse CD3 scFv, grew temporarily in the mice and were subsequently rejected. Cells expressing CD80 grew progressively, although, slower than the nontransfected cells, which is in accordance with previous findings (Chen et al., J Exp Med, 179, 523-532, 1994).

Ten mice were transplanted three times, 7 days apart, with 2×10⁶ of the anti-CD3 (500A2 scFv) transfected cells (without any tumor takes). A control group of 9 mice was injected with PBS only. Subsequently, both groups were challenged with 10⁶ K1735-wt cells. The K1735-WT cells formed tumors in all control mice, but six of the ten immunized mice did not develop tumors. Tumor growth in the four of the immunized mice that developed tumors was delayed compared to that in the non-immunized (control) group. There was no evidence of toxicity or immunosuppression in any of the mice, including mice that had been given anti-CD3 scFv-transfected tumor cells repeatedly.

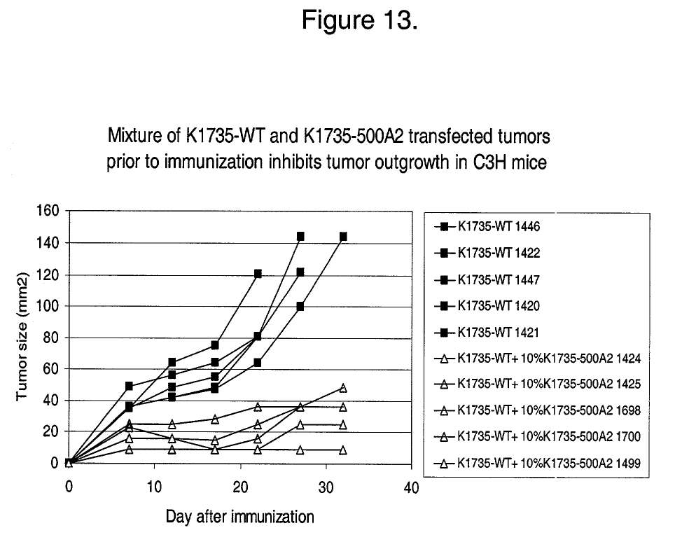

Evidence for a bystander effect when K1735-500A2 cells are admixed to K1735-WT cells. In order to investigate whether tumor cells expressing anti-CD3 scFv in vivo, e.g. as a result of in vivo transfection, would induce an immune response that is effective also against wild type tumor cells, two experiments were performed in which K1735-WT cells were mixed with K1735-500A2 cells. The first experiments showed that when equal numbers of the two cell types were mixed, the tumors regressed after a short period of in vivo growth. In the second experiment, 2×10⁶ K1735-WT cells were mixed with 2×10⁵ K1735-500A2 cells. As shown in FIG. 13, outgrowth of the WT cells was inhibited as compared to that when they were transplanted alone.

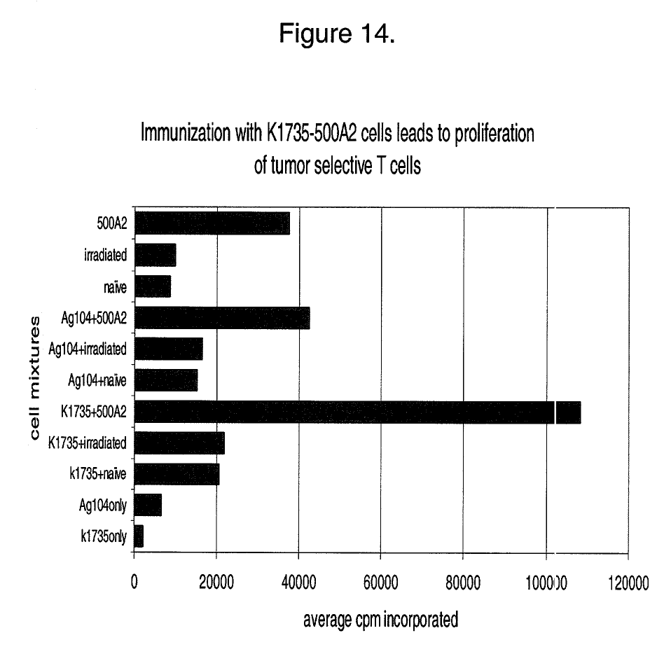

Immunization with K1735-500A2 cells leads to proliferation of tumor-selective T cells. Spleen cells were harvested from mice that had rejected transplanted K1735-500A2 cells. FIG. 14 shows that the proliferation of such spleen cells, when combined with irradiated K1735-WT cells in vitro, proliferate to a much larger extent than spleen cells combined with irradiated cells from the antigenically distinct, syngeneic sarcoma Ag104. Spleen cells from mice immunized with irradiated K1735-500A2 cells do not proliferate more than spleen cells from naïve (control) mice.

Conclusions. Expression of anti-CD3 scFv at the tumor cell surface induces rapid killing of the tumor cells, and causes T cell proliferation. These properties promote tumor specific immunity since the destruction of tumor cells and polyclonal activation of T cells generates tumor antigens that are taken up by dendritic cells maturing under the influence of cytokines produced by the T cells. T cells are first sensitized by the polyclonal anti-CD3 activation, and then tumor specific T cells continue to expand as they recognize tumor antigens presented by APC. Type 1 lymphokines formed by the activated T cells, as well as the T cells themselves, can destroy bystander tumor cells, indicating that transfection of tumor cells, in vivo, to express anti-CD3 scFv can be therapeutically efficacious.

Anti-4-1BB scFv for gene therapy of cancer. Monoclonal antibodies to 4-1BB are effective for therapy of established mouse tumors (Melero, Shuford et al., Nat Med, 3, 682-5, 1997). To construct a vaccine that stimulates the immune system similar to an efficacious MAb, we constructed a vector encoding cell-bound single chain Fv fragments from hybridoma 1D8 (an anti4-1BB monoclonal antibody) (Melero, Shuford et al., Nat Med, 3, 682-5, 1997) using established techniques (Hayden, Grosmaire et al., Tissue Antigens, 48, 242-54, 1996; Winberg, Grosmaire et al., Immunol Rev, 153, 1996) The vector was transfected into cells from the K1735 melanoma (Ward et al., J.Exp. Med., 170, 1989), selected because of its low immunogenicity and very low MHC class I expression. The transfected cells induce a strong Th1 response, for which CD4⁺, but not CD8⁺, T lymphocytes are necessary and which involves NK cells. Vaccinated mice reject wild type K1735 tumors growing as subcutaneous nodules or in the lung. We postulate that an analogous approach will be effective against micrometastases in human patients, including tumors whose MHC class I expression is very low.

Mice and tumor cell lines. Six to eight-week old female C3H/HeN mice were purchased (Taconic, Germantown, N.Y.). K1735 is a melanoma of C3H/HeN origin from which a metastatic clone, M2, was selected.(Fidler and Hart, Cancer Res, 41, 3266-3267, 1981) In agreement with previous findings, its MHC class I expression was found to be very low (data not shown). Ag104 (Ward, Koeppen et al., J. Exp. Med., 170, 1989) is a spontaneous fibrosarcoma of C3H/HeN originally obtained from Dr H. Schreiber (University of Chicago, Chicago, Ill.). YAC-1 was from American Type Culture Collection (Rockville, Md.). The animal facilities are ALAC approved and our protocols were approved by PNRI's Animal Committee.

Antibodies. R-phycoerythrin (PE)-conjugated MAbs GK1.5 (anti-mouse CD4), 53-6.7 (anti-mouse CD8a) and purified AF3-12.1(anti H-2K^{K}) were from Pharmingen (San Diego, Calif.) and R-PE conjugated goat F(ab′)₂ anti-human IgG from Biosource International (Camarillo, Calif.). MAbs 1694 (anti-CD8) was from Dr R. Mittler (Emory University, Atlanta, Ga.). GK1.5 (anti-CD4) was produced by a hybridoma obtained from ATCC. Rabbit anti-asialo GM1 antibodies came from Wako Pure Chemical Industries, (Richmond, Va.), and purified rat IgG from Sigma and Rockland (Gilbertsville, Pa.)

Vectors and transfection of K1735 cells. Methods of variable region cloning, scFv construction, and generation of scFv expression have been described (Gilliland, Norris et al., Tissue Antigens, 47, 1-20, 1996; Hayden, Grosmaire et al., Tissue Antigens, 48, 242-54, 1996; Winberg, Grosmaire et al., Immunol Rev, 153, 1996). The present studies were performed with variable region genes from the anti-4-1BB hybridoma 1D8 (Melero, Shuford et al., Nat Med, 3, 682-5, 1997) to obtain surface expression of cell-bound 1D8 scFv (Hayden, Grosmaire et al., Tissue Antigens, 48, 242-54, 1996; Winberg, Grosmaire et al., Immunol Rev, 153, 1996). For expression of scFv, the transmembrane domain and cytoplasmic tail from CD80 was used, since it mediates cytoskeletal attachment and crosslinking during cell-cell contact (Doty and Clark, J Immunol, 157, 3270-9, 1996; Doty and Clark, J Immunol, 161, 2700-7, 1998). The scFv gene fusion construct in pLNCX was transfected into RetroPack™ PT67 packaging cells (Clontech Laboratories, Inc, Palo Alto, Calif.) by CaPO₄ precipitation. K1735-WT cells were transfected using medium from those cells. G418 resistant clones were stained by PE labeled goat anti-human IgG for scFv surface expression.

Animal Studies. Mice, 5 or 10/group, were transplanted s.c. on one side of the back with 2×10⁶ K1735-WT or K1735-1D8 cells or with irradiated (12,000 rads) K1735-WT cells . Immunized mice were challenged with K1735-WT (2×10⁶ cells/mouse) or Ag104 (3×10⁵ cells/mouse). Mice with established K1735-WT tumors were transplanted s.c. with K1735-1D8 (2×10⁶ cells/mouse); the immunizing cells were given on the side of the back contralateral to the WT cells. Tumor size was assessed by measuring the two largest perpendicular diameters with calipers and reported as average tumor area (mm²)±SD. Sites where mice were transplanted s.c. were shaved to facilitate tumor measurements.

In one experiment mice were injected i.v. with 3×10⁵ K1735-WT cells in the lateral tail vein to establish pulmonary metastases (Kahn et al., J Immunol, 146, 3235-3241, 1991). Three days later, they were transplanted s.c. on one side of the back with K1735-1D8 cells, and this was repeated weekly for 4 times. Thirty-seven days after transplantation of the WT cells, the mice were sacrificed. India ink (15% in phosphate buffered saline) was injected intratracheally, lungs were removed, and unstained metastases were seen against black normal tissue (Estin et al., Proc Natl Acad Sci U S A, 85, 1052-6, 1988).

In Vivo Depletion of CD4⁺ and/or CD8⁺ T lymphocytes and of NK cells. T cells were depleted as described (Chen, Ashe et al., Cell, 71, 1093-102, 1992), injecting mice i.p. 3 times with MAb to CD4 (GK1.5, rat IgG2b) or CD8 (1694, rat IgG 2a), or with a mixture of the two, at 0.5 mg/mouse for 3 consecutive days. This was followed by 0.5 mg of each MAb every 3 days to maintain the depletion. NK cells were depleted by injections of anti-asialo GM1 antibodies at 30 μl/mouse i.p. every 4 days. On day 12, spleen cells from each group were analyzed by FACS to verify the efficiency of the depletions. Subsequently, the mice were transplanted s.c. with tumor cells.

Proliferation Assays. Spleen cells were seeded into 96-well flat-bottom plates (1×10⁵ cells/well) together with 5×10⁵ syngeneic, irradiated (3,000 rads) spleen cells (as APC) and tumor cells. After incubation for 72 hours, triplicate cultures were pulsed for 16-18 h with 1 μCi ³[H] thymidine (Amersham Pharmacia, Biotech Piscataway, N.J.), the uptake of which was measured.

To investigate which T cells proliferated in vitro, spleen cells were labeled by incubation with 2 μM CFDA SE (5-(and-6)-Carboxyfluorescein diacetate succinimidyl ester) (den Haan, Lehar et al., J. Exp. Med., 192, 1685-1695, 2000) according to the manufacturer's (Molecular Probes, Eugene, Oreg.) protocol, incubated with or without K1735-WT cells for 3 days and analyzed by FACS.

Assay for CTL Activity. Mice were sacrificed 24 weeks after transplantation of K1735-1D8, and spleen cell suspensions prepared. When stated, NK cells were removed using anti-asialo GM1 antibodies plus rabbit complement (Cedarane, Ontario,Canada). 5×10⁶ splenocytes were cultivated for 5 days with 1×10⁵ γ-irradiated (12,000 rads) K1735-WT cells in a 24-well plate (Costar Corp., Cambridge, Mass.). Cytolytic activity was examined in a 4-h Cr⁵¹ release assay at different E/T ratios.

ELISPOT assays. Murine IFNγ ELISPOT kits (R&D Systems, Minneapolis, Minn.) were used according to the manufacturer's protocol, and the plates were counted by Plate-scanning service (Cellular Technology Ltd., Cleveland, Ohio).

Immunohistochemistry. Tissues were removed 10-30 days after tumor injection, fixed in 10% formalin, blocked, sectioned at 4-6 μm and stained using a Vector ABC kit (Vector laboratories, Burlingame, Calif.) according to manufacture's protocol to detect CD4⁺ and CD8⁺ T cells. Sections were also stained with H-E.

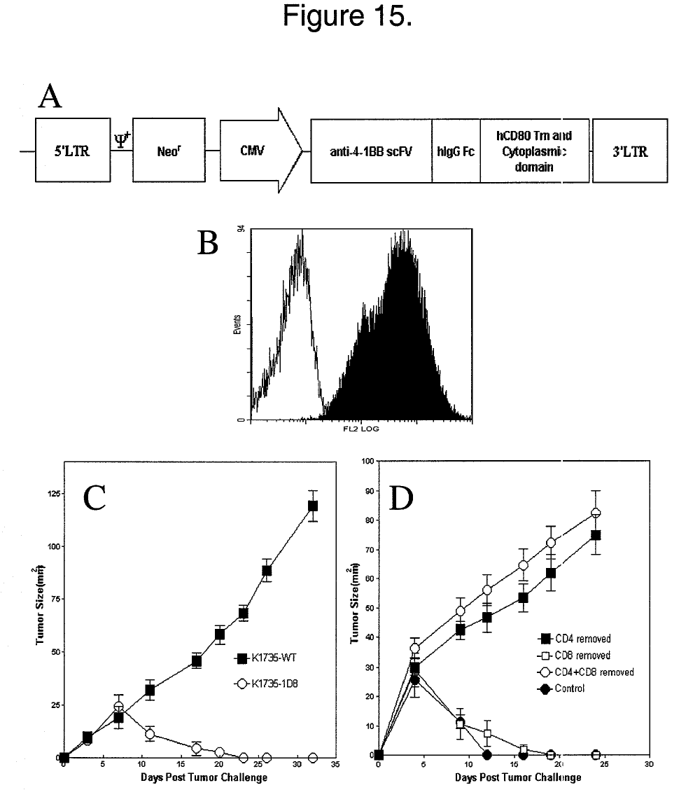

K1735-1D8 cells are rejected through a mechanism that needs CD4⁺ T cells and NK cells. We cloned cell bound anti-4-1BB scFv into a retroviral vector pLNCX (FIG. 15a). The construct was transfected into cells from the metastatic M2 clone of K1735 (Fidler and Hart, Cancer Res, 41, 3266-3267, 1981), referred to as K1735-WT. The transfected line, K1735-1D8, expresses high levels of anti4-1BB scFv at its surface (FIG. 15b).

K1735-WT cells grew progressively when transplanted subcutaneously (s.c.) to naïve syngeneic (C3H) mice. Although the same dose of K1735-1D8 cells initially formed tumors of an approximately 30 mm² surface area, these regressed and had disappeared on day 20 (FIG. 15c). K1735-WT cells transfected with a similarly constructed control vector, which encodes anti-human CD28 scFv, grew in C3H mice at the same rate as K1735-WT cells.

To investigate the roles of CD4⁺ and CD8⁺ T lymphocytes as well as NK cells in the regression of K1735-1D8, we injected naïve mice intraperitoneally (i.p.) with MAbs to remove CD8⁺, CD4⁺ or both CD4⁺ and CD8⁺ T cells or with anti-asialo GM1 rabbit antibodies to remove NK cells. Control mice were injected with rat IgG. Twelve days later, when FACS analysis of spleen cells from similar mice showed that the targeted cell populations were depleted, K1735-1D8 cells were transplanted s.c to each group. K1735-1D8 had similar growth kinetics in mice that had been injected with the anti-CD8 MAb or control rat IgG, while removal of CD4⁺ T cells, alone or together with CD8⁺ T cells, allowed K1735-1D8 to grow equally well as K1735-WT. K1735-1D8 grew in all NK-depleted mice, although more slowly than in the CD4-depleted group (FIG. 15d).

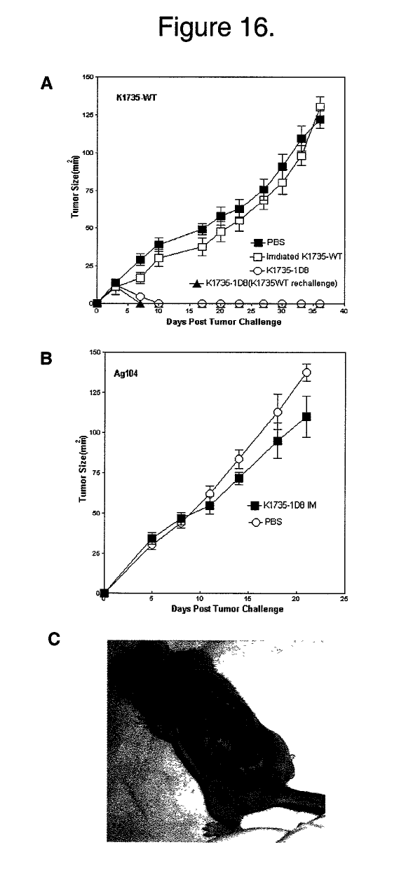

Immunization by K1735-1D8 induces immunity to K1735-WT with memory and specificity. C3H mice, 10 per group, were twice transplanted s.c. at 10 day intervals with either K1735-1D8 or irradiated K1735-WT cells; controls were injected s.c. with PBS. Ten days later, mice were challenged with WT cells. K1735-1D8-immunized mice, but not mice immunized with irradiated K1735-WT, rejected the WT cells (FIG. 16a). One immunization with K1735-1D8 cells was sufficient to protect against transplanted K1735-WT cells.

Two months after rejecting WT cells, mice immunized against K1735-1D8 were again transplanted with WT cells, which were rejected (FIG. 16a). In contrast, cells from the antigenically unrelated sarcoma Ag104 grew as well in the “rejector” mice as in naïve controls (FIG. 16b).

Approximately 20% of the mice twice immunized against K1735-1D8 and subsequently rejecting transplanted WT cells developed depigmentation of the skin which remained during a follow-up period of >4 months (FIG. 16c). There were no other signs of autoimmunity.

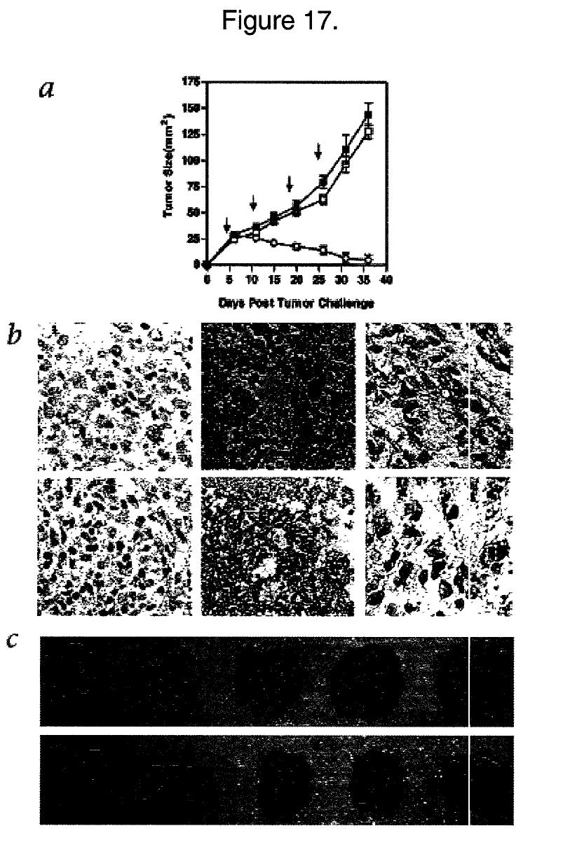

K1735-1D8 cells are effective as a therapeutic vaccine. Three experiments were performed in which mice with established K1735-WT tumors were transplanted with K1735-1D8 cells. The first was performed with mice having s.c. tumors of a surface area of approximately 30 mm². One group was given the first of four weekly injections of K1735-1D8 cells at the side of the back contralateral to the WT tumors. Another group was transplanted with irradiated K1735-WT cells, and a third group received PBS s.c. The WT tumors grew in all control mice and in all mice immunized with irradiated K1735-WT cells. In contrast, they regressed in 4 of the 5 mice immunized against K1735-1D8 (FIG. 17a), which remained tumor-free and without signs of toxicity when the experiment was terminated 3 months later. The tumor nodule in the fifth mouse had decreased in size as long as the mouse received K1735-1D8 cells.

In a second experiment, mice were injected s.c., at weekly intervals, with K1735-1D8, starting 1 day before or either 4 or 8 days after they had been transplanted with K1735-WT cells. For comparison, MAb 1D8 was injected intraperitoneally (i.p.), on the same occasions, to other groups of mice. Controls received PBS i.p. As shown in Table 4, all control mice had to be sacrificed within 49 days of receiving the WT cells because of ≧100 mm² tumors. In contrast, all mice vaccinated with K1735-1D8 cells or given MAb 1D8, starting one day before transplantation of the WT cells, were tumor-free when the experiment was terminated 70 days after transplantation of the WT cells. Mice immunized against K1735-1D8, starting either 4 or 8 days after transplantation of WT cells, had no detectable tumors during the first 28 days of observation, but 4 of those 10 mice developed tumors after the vaccination was discontinued. Mice that were first injected with the MAb 8 days after the WT cells developed tumors earlier than mice in the corresponding K1735-1D8 group, but there was no survival difference between the two groups. Tumors harvested 20 days after transplantation of the WT cells were sectioned and stained with H&E and also evaluated by immunohistochemistry. A tumor nodule from a PBS control mouse comprised many neoplastic cells and a small number of CD4⁺ and CD8⁺ T cells, as did one from a mouse receiving the MAb from day 8. In contrast, a nodule from a mouse first immunized against K1735-1D8 on day 8 contained large numbers of CD4⁺ and CD8⁺ T lymphocytes and only few neoplastic cells (FIG. 17b).

A third experiment, also with 5 mice/group, was performed in which we injected mice intravenously (i.v.) with 3×10⁵ K1735-WT cells to initiate lung metastases. Three days later, K1735-1D8 cells were transplanted s.c., and this procedure was repeated once weekly for a month; control mice were injected with PBS. The experiment was terminated when one mouse in the control group died, 37 days after receiving the WT cells. At that time, lungs of the control mice each had >500 metastatic foci as compared to less than 10 such foci in the lungs from the immunized mice (FIG. 17c).

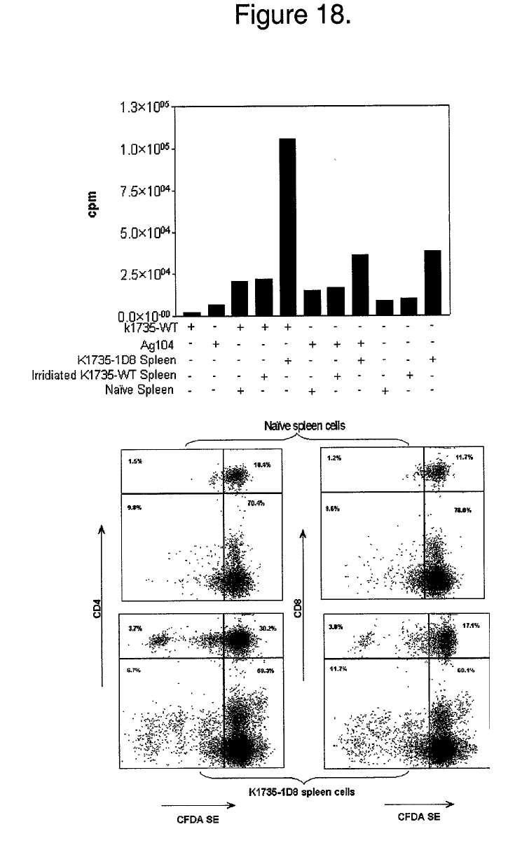

Immunization with K1735-1D8 cells induces a Th1 type immune response. Proliferation of spleen cells, as measured by uptake of tritiated thymidine, from mice immunized against K1735-1D8 was approximately twice that of spleen cells from naïve mice or mice immunized with irradiated K1735-WT (FIG. 18a). It increased almost 4-fold when the spleen cells from K1735-1D8 immunized mice were cultured together with irradiated K1735-WT cells, but not with Ag104 cells. Proliferation assays were also performed in which spleen cells from naïve mice and mice immunized against K1735-1D8 were labeled with CFDA SE (den Haan, Lehar et al., J. Exp. Med., 192, 1685-1695, 2000) before incubation with or without irradiated K1735-WT cells. CD4⁺ and CD8⁺ splenocytes from the K1735-1D8 immune mice proliferated vigorously (FIG. 18b), with the strongest proliferation seen in the presence of K1735-WT cells. Splenocytes from naïve mice did not proliferate.

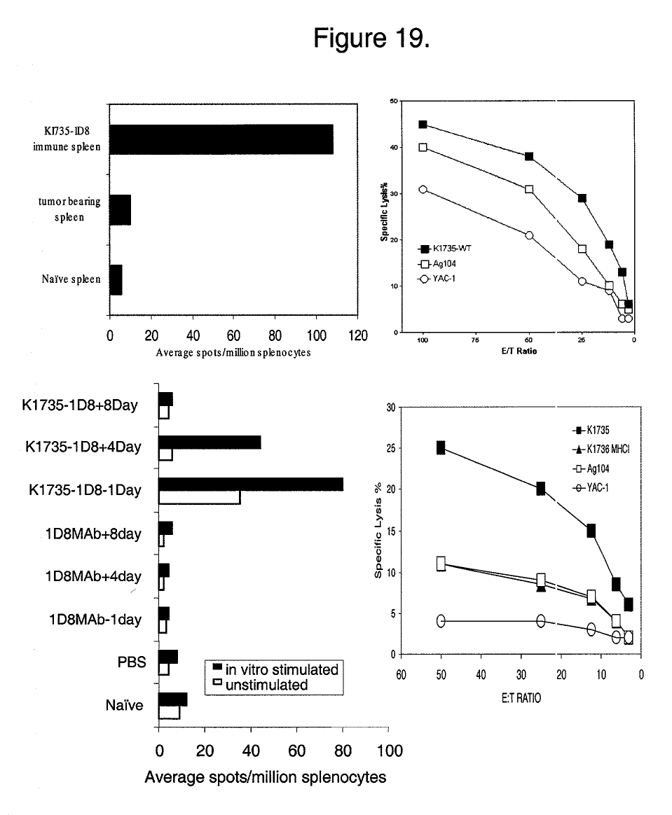

A larger fraction of the spleen cells from mice immunized against K1735-1D8 produced IFNγ in ELISPOT assays than from naïve mice or mice bearing K1735-WT tumors (FIG. 19a). ELISPOT assays with spleen cells from the experiment in Table 4 demonstrated reactivity in mice immunized with K1735-1D8 either one day before or 4 days after transplantation with K1735-WT, and reactivity was higher when the splenocytes were first cocultivated with K1735-WT cells for 3 days (FIG. 19b). The highest reactivity in the group immunized one day before the WT cells may be due to a smaller tumor burden. No reactivity was seen with splenocytes from mice injected with anti4-1BB MAb or with naïve splenocytes.

Spleen cells from mice immunized against K1735-1D8 were incubated with irradiated K1735-WT cells for 5 days and subsequently tested in 4-h Cr⁵¹ release assays. Without prior removal of NK cells, K1735, Ag104 and YAC cells were lysed approximately equally well (FIG. 19c). However, if the spleen cells were first incubated with rabbit anti-asialo GM1 antibodies plus complement to remove NK cells, there was a significant, albeit low, CTL activity against K1735-WT, as compared to Ag104 or YAC, and it could be inhibited by anti-MHC class I MAb (FIG. 19d).

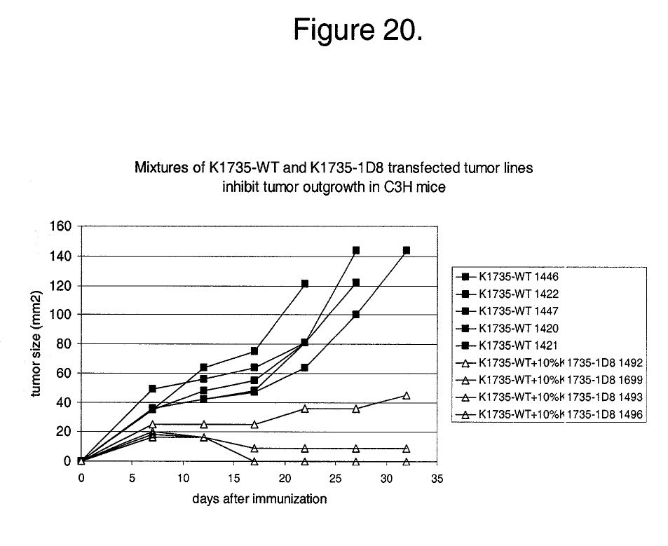

Evidence for a bystander effect when K1735-1D8 cells are admixed to K1735-WT cells. In order to investigate whether tumor cells expressing anti-4-1BB scFv in vivo, e.g. as a result of in vivo transfection, would induce an immune response that is effective also against wild type tumor cells, two experiments were performed in which K1735-WT cells were mixed with K1735-1D8 cells. The first experiments showed that when equal numbers of the two cell types were mixed, the tumors regressed after a short period of in vivo growth. In the second experiment, 2×10⁶ K1735-WT cells were mixed with 2×10⁵ K1735-1D8 cells. As shown in FIG. 20, outgrowth of the WT cells was inhibited as compared to that when they were transplanted alone.

We conclude that K1735-1D8 cells, which express a cell-bound scFv from the anti-4-1BB hybridoma 1D8, are rejected by syngeneic mice, and that CD4⁺ T cells and NK cells, but not CD8⁺ T cells, are necessary for the rejection. We further conclude that immunization against K1735-1D8 induces a systemic immune response to K1735-WT that has both memory and specificity. In contrast, repeated immunization of mice with irradiated K1735-WT cells did not protect against challenge with WT cells, which is consistent with earlier data showing that K1735 has low immunogenicity, even after transfection to express CD80. In vitro assays showed that splenocytes from mice immunized against K1735-1D8 cells. Vaccination of tumor-bearing mice had therapeutic efficacy, both when the tumors grew subcutaneously and in the lung.

The therapeutic efficacy observed against K1735-WT, a tumor of low immunogenicity and very low MHC class I expression should encourage clinical trials in which tumor cells are transfected to express anti-(human) 4-1BB scFv and used as autologous or allogeneic vaccines to destroy micrometastases remaining after cancer patients have received conventional therapy.

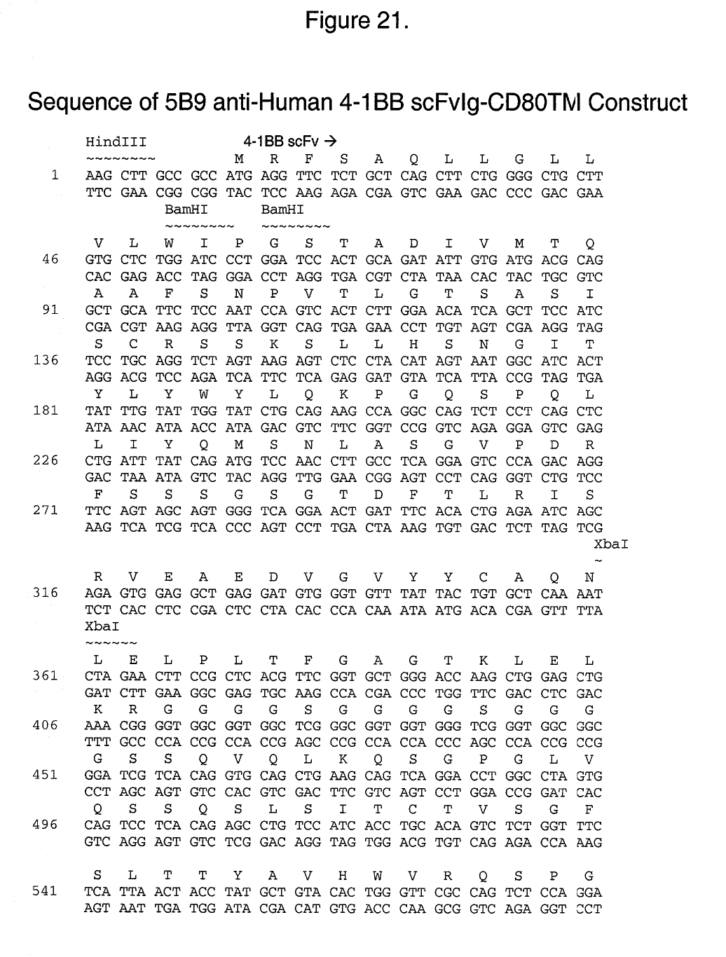

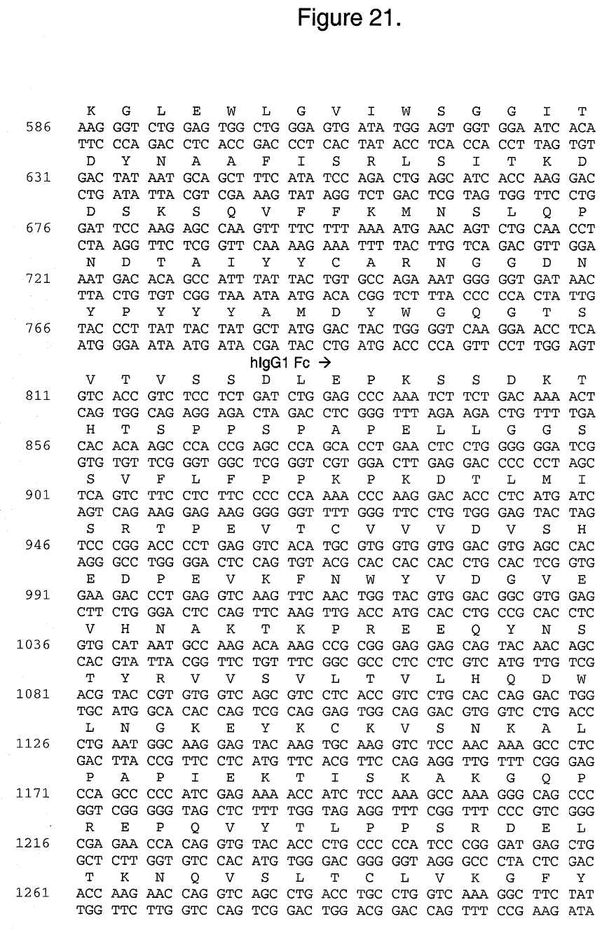

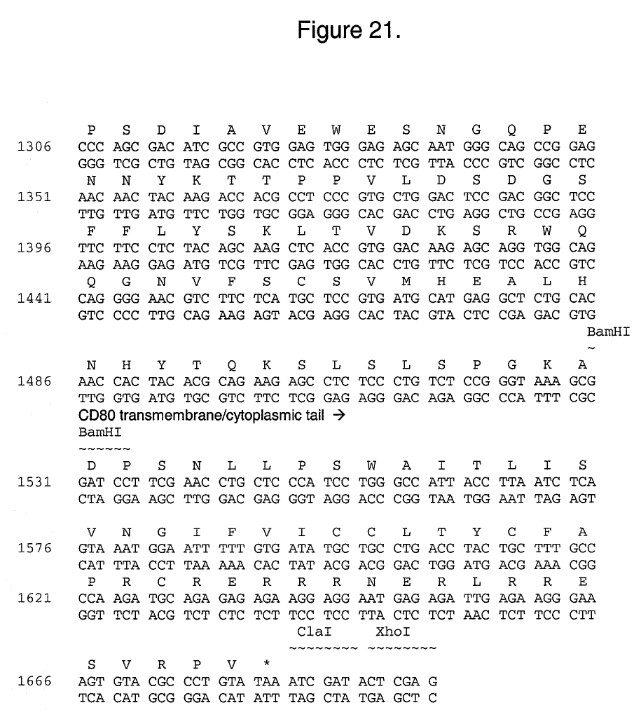

A scFv specific for human 4-1BB was generated from hybridoma 5B9, provided by Dr R. Mittler, Emory University, according to the procedures described above for G194, 500A2 and 1D8 scFv's. FIG. 21 shows the sequence of the 5B9 scFv fused to human IgG1 hinge, CH2, and CH3 domains and the transmembrane domain and cytoplasmic tail from human CD80.

### FIGURE LEGENDS

FIG. 1. Proliferation of in vitro expanded tumor infiltrating lymphocytes (TILs) isolated from a patient with advanced ovarian carcinoma (OV44). Panel A shows lymphocyte proliferation stimulated with control beads plus autologous tumor cells; panel B shows lymphocyte proliferation after stimulation with anti-CD3/CD28/CD40 conjugated beads plus autologous tumor cells; panel C shows lymphocyte proliferation after stimulation with anti-CD3/CD28/CD40 beads without addition of tumor cells.

FIG. 2. Proliferation of peripheral blood lymphocytes from cancer patient 1C after 5 days of in vitro stimulation in the presence of autologous (1C) or allogeneic (4007) ovarian carcinoma cells. Stimulations were; A=control beads; B=anti-CD28/CD40 conjugated beads; C=anti-CD3/CD28 conjugated beads; D=anti-CD3/CD28/CD40 conjugated beads; E=anti-CD3/CD40 conjugated beads.

FIG. 3. Cell mediated cytotoxicity of PBL from patient 1C, tested on the indicated target cells, following PBL activation by autologous tumor cells plus anti-CD3/CD28 beads (A) or by autologous tumor cells plus anti-CD3/CD28/CD40 beads (B).

FIG. 4. IFNγ produced by PBL from patient 1HN with advanced head and neck carcinoma. IFNγ levels in the culture supernatants were measured at different times after stimulation as indicated with anti-CD3/CD28 alone (A), anti-CD3/CD28 beads plus autologous tumor cells (B), or control beads plus autologous tumor cells (C).

FIG. 5. Expression of CD83 on PBMC from a patient with advanced cancer (38C). Cells were stained with anti-CD83 (directly conjugated with phycoerythrin (PE) before stimulation (day 0) or 1 day after stimulation with anti-CD3/CD28 beads.

FIG. 6. PBMC from a patient with advanced cancer (38C) were not activated, or were activated for 2 days with anti-CD3/CD28/CD40 beads, or were activated for 2 days with anti-CD3/CD28 beads as indicated. Cells were stained simultaneously with fluorescein-conjugated anti-CD3 and with PE-conjugated anti-CD83.

FIG. 7. Regression of K1735 melanoma cells transfected to express anti-CD3 scFv (500A2) at their cell surface as compared to K1735 cells expressing murine CD80 and wild type K1735 cells.

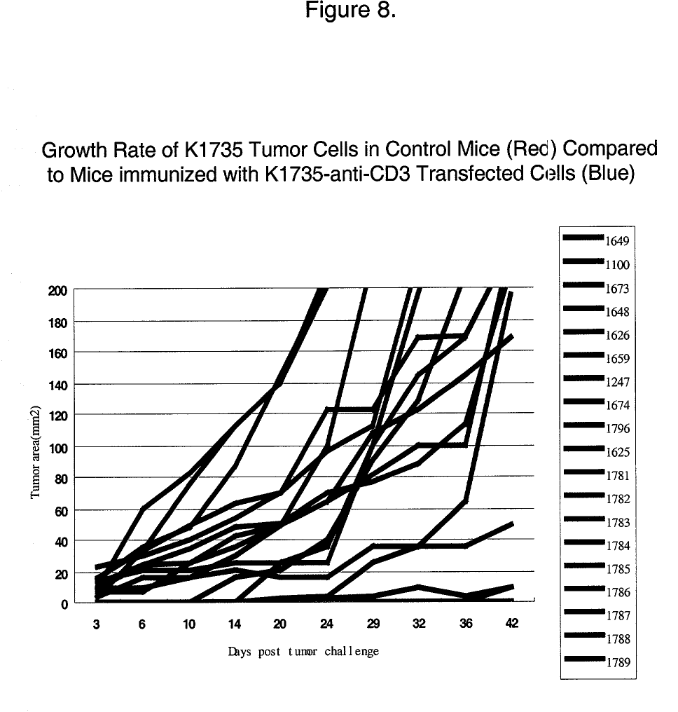

FIG. 8. C3H/HeN mice were immunized three times with K1735M2/500A2 cells (2×10⁶/mouse) at 7 day intervals and then challenged with K1735M2 wild type cells at 1×10⁶/mouse s.c (blue lines). C3H/HeN mice were immunized with PBS then also challenged with K1735M2 wild type at same dose (red lines).

FIG. 9. Sequence of the anti-human CD3 scFv-hIgG1-CD80™ synthetic gene that encodes a product expressed at the cell surface.

FIG. 10. Anti-human CD3 scFv (G194) was expressed on the surface of Reh and T51 cell lines by retroviral gene transduction. Surface expression of the G19-4 scFv gene product was detected using fluorescein-conjugated anti-human IgG to detect the IgG1 CH2 and CH3 domains contained in the gene product. The reaction with wild-type cells in each panel is shown by the dashed line and the reaction with the transfected cells is shown by the solid line.

FIG. 11. Proliferation of T cells induced by culture with Reh or T51 cells expressing anti-CD3 scFv at the cell surface, but not by wild type Reh or T51 cells. Proliferation was measured by uptake of ³H-thymidine during the last 8 hours of a three day culture.

FIG. 12. Resting PBMC rapidly kill Reh and T51 cells expressing anti-CD3 scFv at the tumor cell surface, but do not kill wild type Reh or T51 cells. ⁵¹Cr-labeled cell lines were incubated with PBMC in triplicate cultures at the cell ratios indicated for 8 hours, and the released ⁵¹Cr was measured. Percent specific killing was determined by the classical formula (experimental release minus spontaneous release, divided by maximum release minus spontaneous release).

FIG. 13. Mixtures of K1735-500A2 cells with K1735-WT cells (a proportion of 1:10) are inhibited from outgrowth in immunocompetent syngeneic (C3H) mice. K1735-WT cells were immunized alone or the K1735-WT cells were mixed with K1735-500A2 transfected cells at a 10:1 ratio of untransfected to transfected tumor cells. 2×10⁶ K1735-WT cells were mixed with 2×10⁵ K1735-500A2 cells and the mixed cells used to immunize C3H mice s.c. Tumor growth was monitored at 5 day intervals.

FIG. 14. Splenocytes from mice immunized with K1735-500A2 cells proliferate when combined with irradiated K1735-WT cells but not when combined with Ag104 cells.

FIG. 15. K1735-1D8 cells transplanted to C3H mice are rejected by a CD4⁺ T cell and NK cell dependent mechanism. a) Structure of a retroviral vector containing scFv DNA from the anti-murine 4-1BB hybridoma 1D8; b) Expression of 1D8 scFv on the surface of K1735-1D8 cells, detected by PE conjugated F(ab′)² from goat-anti-human Ig that recognizes the immunoglobulin tail expressed on K1735-1D8 cells (shaded area) but not on K1735-WT cells (solid line); c) Growth kinetics of K1735-1D8(∘) and K1735-WT(▪) cells in naïve mice; d) Growth kinetics of K1735-1D8 cells in mice which had been depleted of CD4⁺(▪), CD8⁺(□), CD4⁺ plus CD8⁺(∘) T cells or of NK(Δ) cells, and in control(●) mice which were injected with purified rat IgG.

FIG. 16. Immunization with K1735-1D8 cells, but not with irradiated K1735-WT cells, protects against outgrowth of transplanted K1735-WT cells by a mechanism that has memory and specificity. a) Mice (10/group) were immunized twice at 10 day intervals by s.c. transplantation of K1735-1D8(∘) or irradiated K1735-WT(□) cells, or they were injected with PBS (▪). Ten days after the last immunization, they were challenged with K1735-WT cells and mice immunized against K1735-1D8 rejected a second challenge of WT cells given 2 months later(▾); b) Ag104 cells were transplanted, 3×10⁵/mouse, to mice that had been immunized against K1735-1D8(▪) and twice rejected K1735-WT cells and to control(∘) mice injected with PBS; c) Depigmentation of skin on the back of a mouse that had been immunized against K1735-1D8 and had rejected K1735-WT cells.

FIG. 17. Therapy of established K1735-WT tumors using K1735-1D8 as immunogen. a) Mice with K1735-WT tumors of 30 mm² surface area that had been transplanted 6 days earlier were vaccinated by s.c. injection of K1735-1D8(∘) or irradiated K1735-WT(□) cells on the contralateral side; mice injected s.c. with PBS(▪) were included as controls. The same treatment was repeated at the indicated (↓) time points; b) Immunohistochemistry of tumors harvested 20 days after transplantation of K1735-WT cells to mice that were untreated (left panel), or first vaccinated with K1735-1D8 8 days after receiving WT cells i-(mid panel),or first injected with MAb 1D8 8 days after receiving WT cells (right panel). The upper and lower areas of each photograph represent CD4⁺T cells and CD8⁺ T cells , respectively, of the same tumor nodules; c) Pulmonary metastases in mice injected i.v. with K1735-WT cells. The upper panel shows lungs from control mice and the lower panel lungs from mice vaccinated by repeated transplantation of K1735-1D8 cells.

FIG. 18. Proliferation of splenocytes from K1735-1D8 immunized mice. a) Spleen cells from mice immunized twice with either K1735-1D8 or irradiated K1735-WTcells were co-cultured with irradiated K1735-WT (“K1735”) or Ag104 (“Ag104”) cells for 3 days; spleen cells from naïve mice were included as a control. Proliferation of tritium labeled spleen cells was measured; b) flow cytometry analysis of the proliferation of CD4⁺ and CD8⁺ spleen cells that had been labeled with CFDA SE.

FIG. 19. INFγ secretion and cytotoxic activity. a) Direct INFγ ELISPOT assay of spleen cells from naïve mice twice immunized with K1735-1D8 cells at 10 day intervals. Spleen cells harvested 10 days after the last immunization were added onto an IFN-γ ELISPOT plate which was incubated for 24 hours; spleen cells from naïve mice and from mice bearing a K1735-WT tumor were tested for comparison; b)In vitro stimulated(▪)and unstimulated(□) spleen cells from the experiment summarized in Table 1 were tested for INFγ secretion in an ELISPOT assay performed 30 days after challenge with K1735-WT. The average number of spots per group (3 replicates) is shown (from top to bottom) for mice given: K1735-1D8 cells one day before, or 4 or 8 days after WT cells; MAb 1D8 one day before, or 4 or 8 days WT cells. The bottom two rows give ELISPOT data for spleen cells from the control group and for spleen cells from naïve mice; c) Splenocytes from K1735-1D8 immunized mice were co-cultured with irradiated K1735-WT cells for 5 days and tested in a 4 hr Cr⁵¹ release assay for lysis of K1735-WT(▪), Ag104(□) and YAC-1(∘) cells. d) An experiment was performed similar to that in FIG. 5c except that the spleen cells had been incubated with anti-asialo GM1 antibodies and rabbit complement to remove NK cells prior to culturing with irradiated K1735-WT cells and testing. Their cytolytic activity against K1735-WT(▪) cells was inhibited by anti-MHC class I Mab(▾). A low cytolytic activity was detected against Ag104(□),while YAC-1(∘) cells were not lysed.

FIG. 20. Mixtures of K1735-1D8 cells with K1735-WT cells (a proportion of 1:10) are inhibited from outgrowth in immunocompetent syngeneic (C3H) mice. K1735-WT cells were immunized alone or the K1735-WT cells were mixed with K1735-1D8 transfected cells at a 10:1 ratio of untransfected to transfected tumor cells. 2×10⁶ K1735-WT cells were mixed with 2×10⁵ K1735-1D8 cells and the cells used to immunize C3H mice s.c. Tumor growth was monitored at 5 day intervals.

FIG. 21. Predicted nucleotide and amino acid sequence of cell surface expressed 5B9 ]g.

### CONCLUSION, RAMIFICATIONS AND SCOPE

The cDNA encoding anti-CD3 or anti4-1BB scFv molecules of the invention that are expressed at the cell surface can be delivered to cancer patients as a DNA plasmid, or can be delivered in a vector such as a viral or bacterial vector. The cDNA encoding anti-CD3 scFv or anti4-1BB expressed at the cell surface can be introduced into cancer cells in vitro, and the gene transduced cancer cells can be used for therapy. Either autologous or allogeneic tumor cells can be used. Combinations of scFv genes encoding molecules expressed at the cell surface are envisioned by the invention, whereby the combinations are chosen from scFv genes that encode scFv molecules that bind to receptors on T cells that provide activation or costimulatory signals. Additional methods for delivery of polyclonal activation signals to T cells in vivo are envisioned by the invention, including injection into patients of slow release polymers containing antibodies to or ligands for surface receptors expressed by T cells.

The invention demonstrates novel methods to generate/expand tumor-selective T lymphocytes in vitro to be used, e.g., for adoptive transfer to patients with cancer, by activating them in the presence of autologous tumor cells and signals via CD3 and costimulatory molecules. The invent on facilitates the in vitro generation of dendritic cells that can present antigen released from the tumor cells so as to expand pre-existing tumor-reactive T cell populations and facilitate the generation of an immune response to antigens that have not been previously recognized. The T cells generated in vitro, as described in the invention, are less sensitive to inhibition by TGF-beta and have a long life-span. The invention also describes two novel types of human tumor vaccines based on the transfection of scFv genes encoding antibody-derived molecules that recognize either CD3 or 4-1BB, and shows that these vaccines can induce tumor-destructive immune responses when tested against a mouse melanoma that has very low immunogenicity and expresses very low levels of MHC class I and no MHC class II. The approach described in the invention can be applied to transfect human tumor cells to express anti-human CD3 or anti-human 4-1BB scFv for use as cell-based vaccines. Furthermore, it can be easily applied to construct gene-based tumor vaccines, in which genes encoding tumor epitopes are combined with genes encoding either anti-CD3 scFv or anti4-1BB scFv, or both. This invention can be expanded by combining scFv's that recognize additional or different immunostimulatory receptors and/or with genes that encode lymphokines that upregulate anti-tumor immune responses.

### Table 1

### Sheet 26 of 29

TABLE 1
Proliferation (cell numbers × 10⁶/sample) and lymphokine production (pg/ml of
TNF or IFNγ) of freshly harvested PBL and TIL from patients with advanced cancer after
culturing for 4-5 days +/− autologous tumor cells in the presence of MAb-conjugated
beads, followed by 2-4 days without beads (same time within each experiment). Cultures
were initiated with 10⁶ PBL or TIL/sample.

| | PBL | | | | | | | | TIL | | | | |
|---|---|---|---|---|---|---|---|---|---|---|---|---|---|
| | 1 OV | | | 8 OV | | 1 HN* | | 480 V* | 30 V | | | 22 C* | |
| MAb-Conjugated Beads | ×10⁶ | TNF | 1FNγ | ×10⁶ | TNF | ×10⁶ | TNF | ×10⁶ | ×10⁶ | TNF | 1FNγ | ×10⁶ | TNF |
| Control | 1.1 | 30 | 479 | 4.2 | 12 | 1.3 | 3 | 0.4 | 1.2 | 0 | 271 | 1.4 | 5 |
| Anti-CD3 | 11.3 | 2440 | 5560 | 15.3 | 1550 | 4.8 | 2080 | NT | 8.2 | 950 | 3720 | NT | NT |
| Anti-CD3/CD28 | 9.6 | 2500 | 7810 | 22.7 | 2500 | 7.2 | 13020 | NT | 7.0 | 2100 | 4730 | 5.8 | 1660 |
| Anti-CD3/CD40 | 7.8 | 2500 | 4480 | 18.6 | >1000 | 4.0 | 1450 | NT | 5.4 | 660 | 3060 | 3.6 | 1540 |
| Anti-CD3/CD28/CD40 | NT | NT | NT | NT | NT | NT | NT | 17.1 | NT | NT | NT | 6.5 | 2060 |

*Autologous tumor cells present together with the lymphocytes
NT = not tested

### Table 2

### Sheet 27 of 29

TABLE 2
CD expression (M ± SD) of PBL from 6 healthy adults and from cancer patients
(5-8 patients per group), tested directly (“unstimulated”) or after culturing with MAb-conjugated beads for
4-5 days. Five to 8 samples tested per group of cancer patients.

| | | Cancer Patients | | | | |
|---|---|---|---|---|---|---|
| | | | Beads | | | |
| | | | | | | Anti- |
| | Healthy Donors | | | | | CD3/CD28/ |
| Marker | Unstimulated | Unstimulated | Control | Anti-CD3/CD28 | Anti-CD3/CD40 | CD40 |
| CD3 | 72.3 ± 11 | 20 ± 24^{Δ} | 52 ± 32 | 92 ± 10* | 96 ± 5* | 94 ± 5* |
| CD4 | 45.4 ± 11 | 21 ± 20 | 37 ± 24 | 42 ± 23 | 29 ± 20 | 51 ± 18 |
| CD8 | 21.7 ± 10 | 9 ± 7 | 18 ± 13 | 47 ± 26 | 70 ± 15* | 45 ± 15* |
| CD28 | 62.1 ± 12 | 33 ± 17^{Δ} | 45 ± 32 | 79 ± 30 | 70 ± 36 | 93 ± 3* |
| CD56 | 2.2 ± 3 | 11 ± 12 | 25 ± 34 | 2 ± 4 | 3 ± 2 | 1.5 ± 2.4 |
| CD80 | 0.1 ± 0 | 2 ± 2 | 4 | 1 ± 1 | 3 ± 5 | 5.6 ± 9 |
| CD86 | 0.2 ± 0 | 34 ± 24^{Δ} | 10 ± 13 | 10 ± 14 | 16 ± 16 | 5 ± 3 |

^{Δ}p < 0.01 compared to unstimulated lymphocytes from healthy donors
*p < 0.01 compared to unstimulated lymphocytes from patients

### Table 3

### Sheet 28 of 29

TABLE 3
Proliferation (cell numbers × 10⁶/sample) and lymphokine production (pg/ml of TNF or IFNγ)
by fresh or previously stimulated (*) PBL and TIL from cancer patients
cultured for 4-5 days with tumor cells and MAb-conjugated beads +/− (5 ng/ml) TGF-β1,
followed by 2-3 days without beads or tumor cells but with TGF-β1 remaining. Cultures
were initiated with 10⁶ PBL or TIL/sample.

| | | PBL | | | | | | | | TIL | | |
|---|---|---|---|---|---|---|---|---|---|---|---|---|
| MAb-Conjugated | TGF-β1 | 22 C | | | 48 OV | | 1 HN* | | | 48 OV | | 22C* |
| Beads | Present | ×10⁶ | TNF | 1NFγ | ×10⁶ | 1FNγ | ×10⁶ | TNF | 1FNγ | ×10⁶ | 1FNγ | ×10⁶ |
| Control | − | 4.6 | 40 | 264 | 0.5 | 310 | 1.3 | 3 | 23 | 4.4 | 98 | 1.7 |
| | + | 2.8 | 0 | 19 | 0.5 | 19 | 0.4 | 5 | 48 | 2.8 | 27 | 1.4 |
| Anti-CD3/CD28/ | − | 19.7 | 2900 | >20000 | 17.2 | >10000 | 7.2 | 11680 | 24050 | 8.1 | 9810 | 6.5 |
| CD40 | + | 15.5 | 860 | 6700 | 9.8 | 3810 | 7.4 | 4640 | 19250 | 3.7 | 5220 | 3.1 |

### Table 4

### Sheet 29 of 29

TABLE 4
Treatment of K1735-WT tumors by vaccination with K1735-1D8 or
injection of Mab 1D8

| | Mean tumor | | | |
|---|---|---|---|---|
| | surface area (mm²) | | | |
| Treatment | 2 weeks | 3 weeks | 4 weeks | Deam from tumor on day |
| PBS(control) | 16 | 37 | 68 | 21,28,45,45,49 |
| K1735-1D8 −1d | 0* | 1* | 2* | _,_,_,_,_ |
| K1735-1D8 +4d | 2* | 1* | 3* | 63,_,_,_,_ |
| K1735-1D8 +8d | 4* | 2* | 2* | 50,50,63,_,_ |
| 1D8 Mab −1d | 2* | 1* | 2* | _,_,_,_,_ |
| 1D8 Mab +4d | 3* | 4* | 5* | 63,63,_,_,_ |
| 1D8 Mab +8d | 11 | 18 | 23 | 28,63,_,_,_ |
| Mean tumor surface area after s.c. transplantation of mice (5/group). Survival recorded over 70 days after transplantation of the WT cells; surviving mice were tumor-free. Starting 1 day before transplantation of the WT cells (“−1d”) or either 4 (“+4d) or 8 (“+8d”) after it, the mice were injected i.p., once a week, with PBS (0.1 ml/mouse as control) or with anti-4-1BB MAb 1D8 (300 | | | | |
| # μg in 0.1 ml volume/mouse) or they were transplanted s.c. on the contra-lateral side of the back with K1735-1D8 cells. *Statistically different from the PBS (control group) P < 0.01 | | | | |

### References

Allison, J. P., C. Chambers, et al. (1998). “A role for CTLA-4-mediated inhibitory signals in peripheral T cell tolerance?” Novartis Found Symp 215: 92-8.

Beatty, P. G., J. A. Ledbetter, et al. (1983). “Definition of a common leukocyte cell-surface antigen (Lp95-150) associated with diverse cell-mediated immune functions.” J Immunol 131(6): 2913-8.

Bennett, M. W., J. O'Connell, et al. (1998). “The Fas counterattack in vivo: apoptotic (depletion of tumor-infiltrating lymphocytes associated with Fas ligand expression by human esophageal carcinoma.” J Immunol 160(11): 5669-75.

Boise, L. H., A. J. Minn, et al. (1995). “CD28 costimulation can promote T cell survival by enhancing the expression of Bcl-XL.” Immunity 3(1): 87-98.

Boon, T., P. G. Coulie, et al. (1997). “Tumor antigens recognized by T cells.” Immunol Today 18(6): 267-8.

Cassian, Y., P. A. Savage, et al. (1999). “Isolation of high avidity melanoma-reactive CTL from heterogeneous populations using peptide-MHC tetramers.” J.Immunol. 162(2227-2234).

Chappell, D. B. and N. P. Restifo (1998). “T cell-tumor cell: a fatal interaction?” Cancer Immunol Immunother 47(2): 65-71.

Cheever, M. A., M. L. Disis, et al. (1995). “Immunity to oncogenic proteins.” Immunol Rev 145: 33-59.

Chen, L., S. Ashe, et al. (1992). “Costimulation of antitumor immunity by the B7 counterreceptor for the T lymphocyte molecules CD28 and CTLA4.” Cell 71(7): 1093-102.

Chen, L., P. McGowan, et al. (1994). “Tumor immunogenicity determines the effect of B7 costimulation on T cell-mediated tumor immunity.” J Exp Med 179: 523-532.

Chen, W., W. Jin, et al. (1998). “Engagement of cytotoxic T lymphocyte-associated antigen 4 (CTLA-4) induces transforming growth factor beta (TGF-beta) production by murine CD4(+) T cells.” J Exp Med 188(10): 1849-57.

Daniel, P. T., A. Kroidl, et al. (1997). “Costimulatory signals through B7.1/CD28 prevent T cell apoptosis during target cell lysis.” J Immunol 159(8): 3808-15.

DeBenedette, M. A., N. R. Chu, et al. (1995). “Role of 4-1BB ligand in costimulation of T lymphocyte growth and its upregulation on M12 B lymphomas by cAMP.” J Exp Med 181(3): 985-92.

den Haan, J. M. M., S. M. Lehar, et al. (2000). “CD8+ but not CD8− dendritic cells cross-prime cytotoxic T cells in vivo.” J. Exp. Med. 192: 1685-1695.

D'Orazio, T. J. and J. Y. Niederkorn (1998). “A novel role for TGF-beta and IL-10 in the induction of immune privilege.” J Immunol 160(5): 2089-98.

Doty, R. T. and E. A. Clark (1996). “Subcellular localization of CD80 receptors is dependent on an intact cytoplasmic tail and is required for CD28-dependent T cell costimulation.” J Immunol 157(8): 3270-9.

Doty, R. T. and E. A. Clark (1998). “Two regions in the CD80 cytoplasmic tail regulate CD80 redistribution and T cell costimulation.” J Immunol 161(6): 2700-7.

Ellenhorn, J. D., R. Hirsch, et al. (1988). “In vivo administration of anti-CD3 prevents malignant progressor tumor growth.” Science 242(4878): 569-71.

Espevik, T. and J. Nissen-Meyer (1986). “A highly sensitive cell line, WEHI 164 clone 13, for measuring cytotoxic factor/tumor necrosis factor from human monocytes.” J Immunol Methods 95(1): 99-105.

Estin, C. D., U. S. Stevenson, et al. (1988). “Recombinant vaccinia virus vaccine against the human melanoma antigen p97 for use in immunotherapy.” Proc Natl Acad Sci U S A 85(4): 1052-6.

Fidler, 1. J. and I. R. Hart (1981). “Biological and experimental consequences of the zonal composition of solid tumors.” Cancer Res 41: 3266-3267.

Finn, O. J., K. R. Jerome, et al. (1995). “MUC-1 epithelial tumor mucin-based immunity and cancer vaccines.” Immunol Rev 145: 61-89.

Fiorentino, D. F., M. W. Bond, et al. (1989). “Two types of mouse T helper cell. IV. Th2 clones secrete a factor that inhibits cytokine production by Th1 clones.” J Exp Med 170(6): 2081-95.

Garlie, N. K., A. V. LeFever, et al. (1999). “T cells coactivated with immobilized anti-CD3 and anti-CD28 as potential immunotherapy for cancer.” J Immunother 22(4): 336-45.

Gilliland, L. K., N. A. Norris, et al. (1996). “Rapid and reliable cloning of antibody variable regions and generation of recombinant single chain antibody fragments.” Tissue Antigens 47(1): 1-20.

Greenberg, P. D. (1991). “Adoptive T cell therapy of tumors: mechanisms operative in the recognition and elimination of tumor cells.” Adv Immunol 49: 281-355.

Hahne, M., D. Rimoldi, et al. (1996). “Melanoma cell expression of Fas (Apo-1/CD95) ligand: implications for tumor immune escape.” Science 274: 1363-1366.

Hayden, M. S., L. S. Grosmaire, et al. (1996). “Costimulation by CD28 sFv expressed on the tumor cell surface or as a soluble bispecific molecule targeted to the L6 carcinoma antigen.” Tissue Antigens 48(4 Pt 1): 242-54.

Hayden, M. S., P. S. Linsley, et al. (1994). “Single-chain mono- and bispecific antibody derivatives with novel biological properties and antitumour activity from a COS cell transient expression system.” Ther Immunol 1(1): 3-15.

Hellstrom, I., K. E. Hellstrom, et al. (1969). “Studies on immunity to autochthonous mouse tumors.” Transplant Proc 1(1): 904.

Hellstrom, K. E. and I. Hellstrom (1969). “Cellular immunity against tumor specific antigens.” Adv Cancer Res 12: 167-223.

Hellstrom, K. E. and I. Hellstrom (1974). “Lymphocyte-mediated cytotoxicity and blocking serum activity to tumor antigens.” Adv Immunol 18: 209-77.

Hellstrom, K. E. and I. Hellstrom (1999). Cancer Vaccines. Handbook of Experimental Pharmacology. P. Perlmann and H. Wigzell. Heidelberg, Springer. Vaccines (Chapter 17): 463-478.

Hu, H. M., W. J. Urba, et al. (1998). “Gene-modified tumor vaccine with therapeutic potential shifts tumor-specific T cell response from a type 2 to a type 1 cytokine profile.” J Immunol 161(6): 3033-41.

Huang, A. Y., P. Golumbek, et al. (1994). “Role of bone marrow-derived cells in presenting MHC class I-restricted tumor antigens.” Science 264(5161): 961-5.

Hurtado, J. C., Y. J. Kim, et al. (1997). “Signals through 4-1BB are costimulatory to previously activated splenic T cells and inhibit activation-induced cell death.” J Immunol 158(6): 2600-9.

June, C. H., J. A. Ledbetter, et al. (1990). “Role of the CD28 receptor in T-cell activation.” Immunol Today 11(6): 211-6.

Kahn, M., H. Sugawara, et al. (1991). “CD4+ T cell clones specific for the human p97 melanoma-associated antigen can eradicate pulmonary metastases from a murine tumor expressing the p97 antigen.” J Immunol 146(9): 3235-3241.

Kehrl, J. H., L. M. Wakefield, et al. (1986). “Production of transforming growth factor beta by human T lymphocytes and its potential role in the regulation of T cell growth.” J Exp Med 163(5): 1037-50.

Kerr, W. G. and J. J. Mule' (1994). “Gene therapy: current status and future prospects.” J. Leuko. Biol. 56: 210-214.

Kiessling, R., K. Kono, et al. (1996). “Immunosuppression inhuman tumor-host interaction: role of cytokines and alterations in signal-transducing molecules.” Springer Semin. Immunopathol. 18: 227-242.

Kiessling, R., K. Wasserman, et al. (1999). “Tumor-induced immune dysfunction.” Cancer Immunol. Immunother. 48: 353-362.

Kim, Y. J., S. H. Kim, et al. (1998). “Human 4-1BB regulates CD28 co-stimulation to promote Th1 cell responses.” Eur J Immunol 28(3): 881-90.

Klein, G., H. O. Sjögren, et al. (1960). “Demonstration of resistance against methylcholanthrene-induced sarcomas in the primary autochthonous host.” Cancer Res 20: 1561-1572.

Krummel, M. F. and J. P. Allison (1996). “CTLA-4 engagement inhibits IL-2 accumulation and cell cycle progression upon activation of resting T cells.” J Exp Med 183(6): 2533-40.

Kugler, A., G. Stuhler, et al. (2000). “Regression of human metastatic renal cell carcinoma after vaccination with tumor cell-dendritic cell hybrids.” Nature Medicine 6: 332-.

Kume, T., K. Oshima, et al. (1999). “Relationship between Fas-ligand expression on carcinoma cell and cytotoxic T-lymphocyte response in lymphoepithelioma-like cancer of the stomach.” Int J Cancer 84(4): 339-43.

Leach, D. R., M. F. Krummel, et al. (1996). “Enhancement of antitumor immunity by CTLA4 blockade.” Science 271(5256): 1734-6.

Ledbetter, J. A., C. H. June, et al. (1986). “Valency of CD3 binding and internalization of the CD3 cell-surface complex control T cell responses to second signals: Distinction between effects on protein kinase C, cyplasmic free calcium, and proliferation.” J. Immunol. 136: 3945-3952.

Ledbetter, J. A., G. Shu, et al. (1987). “Augmentation of normal and malignant B cell proliferation by monoclonal antibody to the B cell-specific antigen BP50 (CDW40).” J Immunol 138(3): 788-94.

Levine, B. L., W. B. Bernstein, et al. (1997). “Effects of CD28 costimulation on long-term proliferation of CD4+ T cells in the absence of exogenous feeder cells.” J Immunol 159(12): 5921-30.

Linsley, P. S. and J. A. Ledbetter (1993). “The role of the CD28 receptor during T cell responses to antigen.” Annu Rev Immunol 11: 191-212.

Loh, E. Y., J. F. Elliott, et al. (1989). “Polymerase chain reaction with single-sided specificity: analysis of T cell receptor delta chain.” Science 243(4888): 217-20.

Maeurer, M. J., S. M. Gollin, et al. (1996). “Tumor escape from immune recognition: lethal recurrent melanoma in a patient associated with downregulation of the peptide transporter protein TAP-1 and loss of expression of the immunodominant MART-1/Melan-A antigen.” J Clin Invest 98(7): 1633-41.

Martin, P. J., J. A. Ledbetter, et al. (1986). “A 44 kilodalton cell surface homodimer regulates interleukin 2 production by activated human T lymphocytes.” J Immunol 136(9): 3282-7.

Matter, B., C. F. Zukoski, et al. (1970). “Transplanted carcinoma in an immunosuppressed patient.” Transplantation 9(1): 71-4.

Melero, I., N. Bach, et al. (1998). “Amplification of tumor immunity by gene transfer of the co-stimulatory 4-1BB ligand: synergy with the CD28 co-stimulatory pathway.” Eur J Immunol 28(3): 1116-21.

Melero, I., J. V. Johnston, et al. (1998). “NK1.1 cells express 4-1BB (CDw137) costimulatory molecule and are required for tumor immunity elicited by anti4-1BB monoclonal antibodies.” Cell Immunol 190(2): 167-72.

Melero, I., W. W. Shuford, et al. (1997). “Monoclonal antibodies against the 4-1BB T-cell activation molecule eradicate established tumors.” Nat Med 3(6): 682-5.

Melief, C. J. and W. M. Kast (1995). “T-cell immunotherapy of tumors by adoptive transfer of cytotoxic T lymphocytes and by vaccination with minimal essential epitopes.” Immunol Rev 145: 167-77.

Miller, A. D. and G. J. Rosman (1989). “Improved retroviral vectors for gene transfer and expression.” Biotechniques 7(9): 980-2, 984-6, 989-90.

Mizoguchi, H., J. J. O'Shea, et al. (1992). “Alterations in signal transduction molecules in T lymphocytes from tumor-bearing mice [see comments].” Science 258(5089): 1795-8.

Mule', J., F. R. Jones, et al. (1979). “Selective localization of radiolabelled immune lymphocytes into syngeneic tumors.” J. Immunol. 123: 600-606.

Nakagomi, H., M. Petersson, et al. (1993). “Decreased expression of the signal-transducing z chains in tumor-infiltrating T cells and NK cells of patients with colorectal carcinoma.” Cancer Res 53: 5610-5612.

Natoli, G., A. Costanzo, et al. (1998). “Apoptotic, non-apoptotic, and anti-apoptotic pathways of tumor necrosis factor signalling.” Biochem Pharmacol 56(8): 915-20.

Nestle, F. O., S. Alijagic, et al. (1998). “Vaccination of melanoma patients with peptide- or tumor lysate-pulsed dendritic cells [see comments].” Nat Med 4(3): 328-32.

Old, L. J. and Y.-T. Chen (1998). “New paths in human cancer serology.” J. Exp. Med. 187: 1163-1167.

Pardoll, D. M. (1996). “Cancer vaccines: a road map for the next decade.” Curr Opin Immunol 8(5): 619-21.

Ranga, U., C. Woffendin, et al. (1998). “Enhanced T cell engraftment after retroviral delivery of an antiviral gene in HIV-infected individuals.” Proc. Natl. Acad. Sci. 95: 1201-1206.

Rodolfo, M., C. Zilocchi, et al. (1999). “IL4-transduced tumor cell vaccine induces immunoregulatory type 2 CD8 T lymphocytes that cure lung metastases upon adoptive transfer.” J Immunol 163(4): 1923-8.

Rosenberg, S. A. (1995). Cell transfer therapy: clinical applications. Biologic Therapy of Cancer (Chapter 19). V. T. DeVita, S. Hellman and S. A. Rosenberg. Philadelphia, Pa., J. P. Lippincott: 487.

Rosenberg, S. A., J. C. Yang, et al. (1998). “Immunologic and therapeutic evaluation of a synthetic peptide vaccine for the treatment of patients with metastatic melanoma [see comments].” Nat Med 4(3): 321-7.

Schoenberger, S. P., L. E. Jonges, et al. (1998). “Efficient direct priming of tumor-specific cytotoxic T lymphocyte in vivo by an engineered APC.” Cancer Res 58(14): 3094-100.

Schwartz, R. H. (1989). “Acquisition of immunologic self-tolerance.” Cell 57(7): 1073-81.

Shiraki, K., N. Tsuji, et al. (1997). “Expression of Fas ligand in liver metastases of human colonic adenocarcinomas.” Proc Natl Acad Sci USA 94(12): 6420-5.

Shuford, W. W., K. Klussman, et al. (1997). “4-1BB costimulatory signals preferentially induce CD8+ T cell proliferation and lead to the amplification in vivo of cytotoxic T cell responses.” J Exp Med 186(1): 47-55.

Sulitzeanu, D. (1993). “Immunosuppressive factors in human cancer.” Adv Cancer Res 60: 247-267.

Takahashi, C., R. S. Mittler, et al. (1999). “Cutting edge: 4-1BB is a bona fide CD8 T cell survival signal.” J Immunol 162(9): 5037-40.

Thompson, C. B., T. Lindsten, et al. (1989). “CD28 activation pathway regulates the production of multiple T-cell-derived lymphokines/cytokines.” Proc Natl Acad Sci USA 86(4): 1333-7.

Tsushima, H., Y. Imaizumi, et al. (1999). “Fas antigen (CD95) in pure erythroid cell line AS-E2 is induced by interferon-gamma and tumor necrosis factor-alpha and potentiates apoptotic death.” Exp Hematol 27(3): 433-40.

Walunas, T. L., C. Y. Bakker, et al. (1996). “CTLA4 ligation blocks CD28-dependent T cell activation.” J Exp Med 183(6): 2541-50.

Walunas, T. L., D. J. Lenschow, et al. (1994). “CTLA4 can function as a negative regulator of T cell activation.” Immunity 1(5): 405-13.

Ward, P. I., H. Koeppen, et al. (1989). “Tumor antigens defined by cloned immunological probes are highly polymorphic and are not detected on autologous normal cells.” J.Exp. Med. 170(217-232).

Wilbanks, G. A., M. Mammolenti, et al. (1992). “Studies on the induction of anterior chamber-associated immune deviation (ACAID). III. Induction of ACAID depends upon intraocular transforming growth factor-beta.” Eur J Immunol 22(1): 165-73.

Wilson, R. E., E. B. Hager, et al. (1968). “Inmunologic rejection of human cancer transplanted with a renal allograft.” N Engl J Med 278(9): 479-83.

Winberg, G., L. S. Grosmaire, et al. (1996). “Surface expression of CD28 single chain Fv for costimulation by tumor cells.” Immunol Rev 153(209).

Yang, G., K. E. Hellstrom, et al. (1995). “Antitumor immunity elicited by tumor cells transfected with B7-2, a second ligand for CD28/CTLA-4 costimulatory molecules.” J Immunol 154(6): 2794-800. Yang, G., M. T. Mizuno, et al. (1997). “B7-negative versus B7-positive P815 tumor: differential requirements for priming of an antitumor immune response in lymph nodes.” J Immunol 158(2): 851-8.

We claim:

1. A culture system for generation of tumor-reactive T lymphocytes comprising four components, incuding a) T cells from a patient with cancer; b) antigen presenting cells; c), autologous or allogeneic tumor cells;, and d) immobilized antibodies to T cell receptors that induce polyclonal activation.

2. The culture system of claim 1 where the antigen presenting cells of component b are autologous monocytes that differentiate during the culture due to the cytokines and activation response of the T cells.

3. The culture system of claim 1 where the autologous or allogeneic tumor cells of component c are transfected to express at least one gene encoding a molecule that stimulates a direct or indirect T cell response.

4. The culture system of claim 1 where the immobilized antibodies of component d bind to CD3 and CD28 receptors.

5. A gene of claim 3 comprising DNA encoding anti-CD3 scFv that is expressed at the cell surface.

6. The gene of claim 3 comprising DNA encodingG19-4 scFv.

7. A gene of claim 3 comprising DNA encoding anti-human 4-1BB scFv at the cell surface.8. A gene of claim 3 comprising DNA encoding 5B9 scFv expressed at the cell surface.

8. Autologous or allogeneic tumor cells transfected with a gene encoding anti-CD3 scFv that is expressed on the tumor cell surface

9. Autologous or allogeneic tumor cells of claim 8 transfected with G19-4 scFv that is expressed on the tumor cell surface

10. Autologous or allogeneic tumor cells transfected with a gene encoding anti-4-1BB scFv that is expressed on the tumor cell surface

11. Autologous or allogeneic tumor cells of claim 10 transfected with 5B9 scFv that is expressed on the tumor cell surface

12. A method for cancer therapy that includes injecting cancer patients with tumor cells transfected to express anti-CD3 scFv at the cell surface

13. A method for cancer therapy of claim 12 that includes injecting cancer patients with tumor cells transfected to express G19-4 scFv at the cell surface.

14. A method for cancer therapy that includes injecting tumor cells transfected to express anti-4-1BB scFv at the cell surface.

15. A method for cancer therapy of claim 14 that includes injecting cancer patients with tumor cells transfected to express 5B9 at the cell surface.

16. A method for cancer therapy that includes injecting cancer patients a with DNA plasmid encoding anti-CD3 scFv at the cell surface.

17. A method for cancer therapy of claim 16 that includes injecting cancer patients with a DNA plasmid encoding G19-4 scFv at the cell surface.

18. A method for cancer therapy that includes injecting cancer patients with a DNA plasmid encoding anti-4-1BB at the cell surface.

19. A method for cancer therapy of claim 18 that includes injecting cancer patients with a DNA plasmid encoding 5B7 scFv at the cell surface.

20. DNA for cancer gene therapy encoding anti-CD3 scFv at the cell surface

21. DNA for cancer gene therapy encoding G19-4 scFv at the cell surface

22. DNA for cancer gene therapy encoding anti-4-1BB at the cell surface

23. DNA for cancer gene therapy encoding 5B9 scFv at the cell surface
